(19) United States
(12) Patent Application Publication
(10) Pub. No.: US 2015/0197783 A1
(43) Pub. Date: Jul. 16, 2015

(54) METHOD FOR THE DEGRADATION OF KERATIN AND USE OF THE KERATIN HYDROLYSATE PRODUCED

(71) Applicant: DUPONT NUTRITION BIOSCIENCES APS, Copenhagen (DK)

(72) Inventors: Shukun Yu, Bunkeflostrand (SE); Charlotte Horsmans Poulsen, Brabrand (DK); Jon Gade Hansted, Aarhus V (DK)

(73) Assignee: DUPONT NUTRITION BIOSCIENCES APS, Copenhagen (DK)

(21) Appl. No.: 14/415,563

(22) PCT Filed: Jul. 19, 2013
(86) PCT No.: PCT/EP2013/065364

§ 371 Date: Jan. 16, 2015

(30) Foreign Application Priority Data
Jul. 20, 2012 (GB) 1212932.6

(51) Int. Cl. C12P 21/06; A23K 1/00; A23K 1/10; A23J 3/34

(52) U.S. Cl. CPC C12P 21/06; A23J 3/34; A23K 1/001; A23K 1/106

(57) ABSTRACT

The present invention relate to a method for degrading keratin comprising the step of admixing at least 5 g of keratin material with a protease and a reducing agent under controlled oxygen levels; as well as keratin hydrolyzate so produced and uses thereof.

### REFERENCE TO A SEQUENCE LISTING

Attached is a sequence listing comprising SEQ ID NOs: 1-4, which are herein incorporated by reference in their entirety.

### FIELD OF THE INVENTION

The present invention relates to methods for degrading keratin or producing keratin hydrolyzate, as well as methods of using the degraded keratin.

### BACKGROUND OF THE INVENTION

Keratin is the collective name for a family of tough proteins which are found in a number of structures. It is a protein used by numerous groups of animals as a structural element, and is a classic example of a fibrous protein. To fulfil this structural function, Keratin molecules are helical and fibrous, twisting around each other to form strands called intermediate filaments. It is thought that this makes a lot of the protein inaccessible to enzyme digestion at first instance. Additionally, keratin proteins contain a high percentage of sulfur-containing amino acids, largely cysteine, which form disulfide bridges between the individual molecules and assist in forming the fairly rigid structure of keratin. Unfortunately, the disulphide bridges also make digestion and degradation of the keratin rather difficult. There are two main types of keratin, alpha- and beta-keratins. The α-keratins are mostly present in the hair (including wool), horns, nails, claws and hooves of mammals. The harder β-keratins are found in nails and in the scales and claws of reptiles, their shells (Testudines, such as tortoise, turtle, terrapin), and in the feathers, beaks, claws of birds and quills of porcupines. β-keratins are formed primarily in beta sheets, although some beta sheets are also found in α-keratins.

The degradation of keratin can be of significant commercial value. One source of keratin in particular—feathers—are produced in vast quantities by the poultry industry. In 2002 approximately 49 billion chickens were utilized in the poultry industry. Poultry feathers typically contain approximately 90% protein in the form of β-keratin. However, keratin must be cleaved before its protein content can be digested by animals (McCasland and Richardson 1966, Poult. Sci., 45:1231-1236; Moran et al. 1966 Poult. Sci., 45: 1257-1266). Degradation of feathers can therefore provide an inexpensive source of digestible protein and amino acids. Accordingly feather hydrolyzate (i.e. degraded feathers) can be utilized in a numbers of ways, such as in animal feed. However, current methods of recovering this nutriment are so inefficient and costly that the vast majority of keratin waste streams are simply disposed of in landfill or via incineration, both of which can cause environmental problems and reduce the sustainability of the main commercial process (often meat production for human consumption). Unfortunately some contemporary processes for the production of keratin hydrolyzate produce feather meal that is more expensive than chicken meat.

Degradation of keratin can be achieved by steam hydrolysis, chemical hydrolysis and enzyme hydrolysis. For example, steam hydrolysis is disclosed in M. J. Considine, 2000, New Enzyme Technologies For Poultry By-Products. Proc. Aust. Poult. Sci. Sym. 2000 . . . 12, pages 163-165, ISSN No. 1034-6260). “Feather meal” is a byproduct of processing poultry; it is made from poultry feathers by partially hydrolyzing them under elevated heat and pressure, and then grinding and drying. Synonyms include “hydrolyzed feather protein”, “feather flour”, and “hydroylzed poultry by-products aggregate”. The most popular method of feather meal production is by hydrothermal process wherein feathers are cooked under high pressure at high temperature (typically around 120-140° C. for 10 to 90 min). One serious disadvantage of, steam hydrolysis is that the process can degrade heat sensitive essential amino acids like methionine, lysine, tyrosine, tryptophan, thereby depleting the nutritional content of the resultant feather hydrolyzate. Furthermore, it has been found that the feather hydrolyzate produced by steam hydrolysis has a relatively low digestibility and low nutritional value (Papadopolous et al. 1986 Animal Feed Sci Technol 14: 279-290; Ellingson T. A. Master thesis 1993, Virginia Tech; Wang X and Parson CM 1997 Poultry Sci 76: 491-496). This is clearly undesirable for the use of the feather hydrolyzate in feed as the amount of protein and amino acids available to the animal is suboptimal.

A number of patents Shih et al. granted in the name of North Carolina State University relate to the use of B. licheniformis PWD-1 or a keratinase isolated from this bacterium to degrade poultry feathers see, U.S. Pat. No. 4,908,220, U.S. Pat. No. 4,959,311, U.S. Pat. No. 5,063,311, U.S. Pat. No. 5,171,682, U.S. Pat. No. 5,186,961 and U.S. Pat. No. 5,712,147. Enzyme degradation can be advantageous as some processes may result in more digestible protein and amino acids being present compared with the use of steam hydrolysis or chemical hydrolysis.

However, as disclosed in Cai et al. (J. Zhejiang Univ. Sci. B 2008 9(9):713-720), the use of a protease (such as keratinase) alone may be very inefficient in the degradation of keratin. Cai et al. disclose the use of reducing agents such as DTT, B-mercaptoethanol, cysteine and sodium sulfite and sulphur-containing chemical such as SDS, ammonium sulfatate and DMSO stimulated feather meal hydrolysis due to the direct breakdown of disulfide bonds by the reducing agents or due to reactions caused by the sulphur-containing chemicals. However, the addition of reduction agents and/or sulfide chemicals can lead to significant cost increases. A further disadvantage of Cai et al. is that many of these reducing agents are sulphur-containing and so although good at disrupting disulfide bridges, they are less appealing for food use (for example, sodium dodecyl sulfate (SDS), dithiothreitol (DTT), mercaptoethanol, L-cysteine, sodium sulphite, ammonium sulfamate and dimethylsulfoxide (DMSO)). Additionally, rather than teaching a process suitable for use on raw plucked feathers, Cai et al. teach the use of these harsh chemicals on feather meal which has already undergone a hydrothermal hydrolysis step prior to the application of enzyme and reducing agents, it being apparent that said hydrothermal hydrolysis would have greatly reduced the nutritional content of the keratin hydrolysate for animals. Hence this process is not considered an ideal solution for the production of keratin hydrolysates most suitable for use in human or animal food. Accordingly, there exists a need for process which can provide a good source of digestible protein and/or amino acids in a more cost-effective manner and preferably with reduced sulphur content.

### BRIEF DESCRIPTION OF THE DRAWINGS

FIG. 1 shows (from left to right) control flasks 1 to 3 and deaerated flasks 4 to 6 following 22 hr at 50° C. Each flask comprised 3 g unprocessed raw chicken feathers, sodium sulphite and the protease Protex P.

FIG. 2 shows unprocessed raw chicken feather (left) and mechanically ground feather (right) in which the rachis and hollow shaft of the feather are in pieces of less than 0.5 cm in length.

FIG. 3 shows a flow chart of a feather hydrolysis process.

FIG. 4 shows an enzyme hydrolysis reactor diagram.

FIG. 5 shows the enzyme hydrolysis followed by 121° C. for 20 min showing that the vanes and barbs are still attached to the rachis and hollow shaft.

FIG. 6 shows Sequence ID NO 1 Protex P

FIG. 7 shows Sequence ID NO 2 Protex 6L

FIG. 8 shows Sequence ID NO 3

FIG. 9 shows Sequence ID NO 4

### SUMMARY OF THE INVENTION

A seminal finding of the present invention is that by controlling oxygen levels a process of preparing keratin hydrolyzate may be more cost-efficient. This finding is particularly surprising as hitherto commercial production of keratin hydrolyzate was not carried out under controlled oxygen levels.

For the first time the present inventors have shown that by controlling oxygen levels a keratin material may be degraded to by a desired amount using less chemical agents. This surprising finding allows keratin to be degraded to provide, and keratin hydrolyzate to be produced which has, a good source of digestible protein and amino acids in a more cost-effective manner. Furthermore, this may advantageously result in less chemicals being present in the final product.

### STATEMENTS OF THE INVENTION

In one aspect, the present invention relates to a method of commercially producing keratin hydrolyzate comprising the step of:
i) admixing at least 1 kg of keratin material with a protease and a reducing agent under controlled oxygen levels.

In a further aspect, the present invention relates to a method of degrading keratin comprising admixing at least 1 kg keratin material with a protease and a reducing agent under controlled oxygen levels.

In another aspect, the present invention relates to a method of producing animal feed comprising admixing keratin hydrolyzate produced by a method of the present invention.

In another aspect, the present invention relates to the use of keratin hydrolyzate produced by a method of the present invention in feed.

In another aspect, the present invention relates to keratin hydrolyzate produced by a method of the present invention.

In a further aspect, the present invention relates to a feed additive composition comprising the keratin hydrolyzate of the present invention.

In another aspect, the present invention relates to a feed comprising the keratin hydrolyzate of the present invention

These and other aspects of the present invention are described in more detail in the detailed disclosure of the preferred embodiments of the invention below.

### DETAILED DISCLOSURE OF THE PREFERRED EMBODIMENTS OF THE INVENTION

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a protease” includes a plurality of such enzymes and reference to “the feed” includes reference to one or more feeds and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

Method

In one aspect, the present invention relates to a method of degrading keratin or producing keratin hdrolyzate comprising admixing a keratin material with a protease and a reducing agent under controlled oxygen levels.

The term “keratin hydrolyzate” as used herein refers to the resultant product following the hydrolysis of a keratin material by e.g. a protease.

The admixture may occur in a vessel or reactor. In one embodiment, the vessel or reactor is a free-fall vessel or reactor. Examples of free-fall vessels include drum mixers and tumble mixers.

Suitably the keratin material and one or more reducing agents may optionally be admixed (simultaneously or sequentially) with one or more further components.

The keratin material, protease and reducing agent are admixed until a desired degree of degradation of the keratin material has occurred (e.g. until the digestibility of keratin material is increased, thereby enriching the concentration of digestible proteins and peptides therein). A person of ordinary skill in the art will readily understand that the optimal time period used will depend on a number of factors such as the temperature and pH used; the degree of cross-linking present in the keratin material to be degraded; whether other components such as non-sulphur containing surfactants, chemical oxidants, acids or alkalis are used in the process and the ratio of reducing agent and/or protease to keratin material for example.

In one embodiment, the keratin material and protease may be admixed for about 30 minutes to about 48 hours. Suitably, the keratin material and protease may be admixed from about 1 hour to about 42 hours; or from about 2 hours to about 36 hours; or from about 3 hours to about 30 hours; or from about 4 hours to about 24 hours; or from about 5 to about 18 hours

In one embodiment, the keratin material, protease and reducing agent may be admixed for about 30 minutes to about 16 hours or from about 30 minutes to about 14 hours; or from about 1 hour to about 12 hours; or from about 1.5 hours to about 10 hours; or from about 2 hours to about 8 hours; or from about 3 to about 7 hours.

Suitably the keratin material, protease and reducing agent may be admixed for at least 30 minutes or at least 45 minutes or at least 1 hour or at least 1.5 hours or at least 2 hours or at least 2.5 hours or at least 3 hours or at least 3.5 hours or at least 4 hours or at least 4.5 hours or at least 5 hours or at least 5.5 hours or at least 6 hours or at least 7 hours or at least 8 hours or at least 9 hours or at least 10 hours.

In one embodiment, the keratin material, protease and emulsifier is admixed for at least 2 hours.

Suitably, the keratin material, protease and reducing agent may be admixed for less than 16 hours, less than 15.5 hours, less than 15 hours, less than 14.5 hours, less than 14 hours, or less than 13 hours.

In one embodiment, the keratin and material, protease and reducing agent may be admixed for less than 16 hours.

In one embodiment, the keratin and material, protease and reducing agent may be admixed for less than 13 hours

In one embodiment, the keratin and material, protease and reducing agent may be admixed for about 1.5 to about 10 hours.

In one embodiment, the keratin material, protease and reducing agent may be admixed until at least 50% by weight of the keratin material is degraded. Optionally, the keratin material, protease and reducing agent may be admixed until at least 55% (suitably at least 60% or at least 70% or at least 80% or at least 90% or 100%) by weight of the keratin material is degraded. Degraded or degradation of feather material by 100% may be defined by the complete detachment of vanes, barbs and after feather from the rachis and hollow shaft; and the fragmentation of the rachis and hallow shaft such that feather meal is produced in one single step.

In one embodiment, the keratin material (e.g. wet feathers) may be processed (e.g. mechanically) into small pieces. The keratin material may be added to a vessel (e.g. at ambient temperature) and heated (for example to 50-80° C.) with the addition of a protease, a chemical and optionally a reducing agent (e.g. sulfite). The vessel may be a closed reactor optionally with reduced airspace to control oxygen levels. The keratin material and protease are reacted for a suitable time (e.g. for 30 min to 48 hours). The vessel may be rotated or the vessel contents may be admixed (e.g. using a propeller at 1-200 rpm). During the reaction process the mixing may cause some oxygen to be continually introduced into to the reaction liquid. Accordingly, the airspace of the vessel may advantageously have lower level of oxygen (e.g. through the use of stream to expel air out of the reactor). The resultant keratin material (e.g. feather meal) may be dried and may be used as feed components. Suitably, such a process has the advantages of reducing energy associated with grinding the keratin material and the utilisation of particular high temperatures (e.g. 120° C.) which can lead to the damage of certain amino acids. Accordingly, the above process may also be modified such that the keratin material added to the vessel can be heated as high as 110° C. in some facilities having this capability. Admixing of the keratin material and protease may be performed by rotating the vessel or the vessel contents may be admixed (e.g. using a propeller at 1-500 rpm).

Suitably the pH during the reaction is within the working range of the protease used. It is a matter of routine to a person of ordinary skill in the art to determine the optimal working range of the protease used and to add buffer to adjust the reaction solution to an appropriate pH. For example, the working range of the protease Protex 30L (available from DuPont Industrial Biosciences ApS) may be from about 5.5 to about 12. When Protex 30 L is used as the protease, the pH during the admixing step may be from about 5.5 to about 12. Suitably, the pH range may be from about 7 to about 11, or from about 8 to about 10. Preferably, when Protex 30L is used the pH is about pH 9.

In one embodiment, the pH used is pH at about the optimal pH for the protease used. For example, the pH may be +/− about 1 pH of the optimal pH of the protease used (e.g. a pH of about 8 to about 10 for Protex 30L). Suitably, the pH may be +/− about 0.5 pH of the optimal pH of the protease used or about the optimal pH.

In another embodiment, the pH used is the optimal pH of the protease.

Suitably the temperature during the reaction is adjusted to be within the working range of the protease used. It is a matter of routine to a person of ordinary skill in the art to determine the optimal working range of the protease used and to carry out the reaction at a desired temperature. For example, the working range of the protease Protex 30L may be from about 30° C. to about 80° C. When Protex 30 L is used as the protease, the temperature during the admixing step may be from about 30° C. to about 80° C. Suitably, the temperature may be from about 40° C. to about 80° C., or from about 50° C. to about 80° C. Suitably, the temperature may be or from about 60° C. to about 80° C. Preferably, when Protex 30L is used the temperature is about 70° C.

Suitable the temperature used may be + and/or −15° C. or + and/or −10° C.; or + and/or −5° C.; or + and/or −4° C.; or + and/or −3° C.; or + and/or −2° C. or + and/or −1° C. of the optimal temperature of the protease used. Preferably the temperature used is about the optimal working temperature of the protease used.

In one embodiment, the temperature used is + and/or −10° C. of the optimal temperature of the protease used.

In another embodiment, the temperature used is + and/or −5° C. of the optimal temperature of the protease used.

In one embodiment, more than one enzyme may be present in the reaction (e.g. admixing) step. Suitably, the temperature, pH and other reaction conditions used are selected to be within the working ranges of the enzymes used. Suitably, the enzymes used (e.g. more than one protease and/or other additional enzymes) are selected which have overlapping working ranges. Preferably, the enzymes are selected to have compatible, preferably similar working ranges.

Suitably, prior to admixing the keratin material with the protease the feather may be sterilised e.g. to reduce and/or prevent bacterial contamination. This sterilization step may be carried out by any available means. For example, fumigation could be achieved by contacting the keratin material with formalin or ethylene oxide gas. Alternatively and/or additionally, steam sterilisation could be achieved by steaming under pressure. Suitably steam sterilisation may be preferable to aid subsequent enzymatic degradation of the keratin material.

In one embodiment, the keratin material is steam sterilised prior to the reaction step. Suitably, the steam sterilisation comprises a step of contacting the keratin to steam for a time and at a temperature sufficient to facilitate the subsequent enzymatic hydrolysis thereof, even if this steam treatment step does not accomplish a complete sterilization of the keratin material. Suitably, the steam sterilisation may comprise contacting keratin material to steam under pressure, in an enclosed chamber, at 80 to 125° C. for at least 2 minutes or at least 5 minutes or at least 10 minutes or at least 15 minutes or at least 20 minutes. Suitably, the steam sterilisation may comprise contacting keratin material to steam under pressure, in an enclosed chamber, at 120 to 125° C. for at least 2 minutes or at least 5 minutes or at least 10 minutes or at least 15 minutes or at least 20 minutes. Suitably, the time and temperature of steam treatment may be less than those employed in commercial steam hydrolysis processes, which employ treatment times of 35 minutes or more at steam pressures of about 35 p.s.i. or more.

In one embodiment of the present invention, the methods of the present invention may comprise a chemical hydrolysis step which may occur prior to, during and/or after the reaction step with protease and/or reducing agent.

In one embodiment, suitably the acid or alkali used in the chemical hydrolysis may provide the means for pH adjustment to the optimal working conditions of the protease.

In one embodiment of the present invention, the methods of the present invention may comprise a chemical hydrolysis step which may occur prior to and/or during the reaction step with protease and/or reducing agent.

In one embodiment of the present invention, the methods of the present invention may comprise a chemical hydrolysis step which may occur after the reaction step with protease and/or reducing agent.

In one embodiment, suitably the acid or alkali used in the chemical hydrolysis may provide the means for pH adjustment to the optimal working conditions of the protease

In one embodiment, chemical hydrolysis may occur after the reaction step with a protease.

The process of the present invention may be carried out as a batch, fed-batch or continuous process.

In one embodiment, the process of the present invention may be carried out in a batch process. Advantageously, a batch process is more easily adapted for controlling oxygen levels.

In one embodiment, the feather hydrolyzate produced by the method of the present invention may be in the form of a precipitate. Preferably the hydrolyzate is filtered. Suitably, particulate matter greater than 1.0 cm (suitably greater than 0.1 cm) is recycled for pre-treatment or hydrolysis.

In one embodiment, the feather hydrolyzate produced by the method of the present invention may be in the form of a solution.

Controlling Oxygen Levels in the Process

In the methods of the present invention oxygen levels may be controlled during the step of admixing the keratin material with a protease and a reducing agent.

In one embodiment, at least the step of admixing a keratin material with a protease and an emulsifier occurs under controlled oxygen levels.

Without wishing to be bound by theory it is thought that by controlling and/or reducing the amount of oxygen available (such as in an open system) the amount of reducing agent may be reduced thereby saving costs and/or providing a safer final product.

By “under controlled oxygen levels” it is meant that the reaction does not have unlimited access to oxygen (such as in an open system). Various means of controlling oxygen levels are known in the art. For example, use of a closed system (e.g. a sealed vessel or reactor) results in a controlled level of oxygen present in the headspace above the reaction solution. Alternatively, means of reducing the level of available oxygen can be employed such as heating the reaction medium, steam flushing, vacuum pumping and/or the addition of nitrogen (e.g. nitrogen bubbling).

In one embodiment, at least the step of reacting a keratin material with a protease and a reducing agent occurs in a closed system (e.g. in a sealed vessel or reactor).

By “under controlled oxygen levels” it is meant that that the reaction does not have unlimited access to oxygen (such as in an open system). Various means of controlling oxygen levels are known in the art. Such methods include heating the reaction medium, vacuum pumping and/or nitrogen bubbling. Suitably, the method of the present invention preferably uses a closed system, addition of nitrogen, vacuum pumping or any combination thereof to control oxygen levels.

In one embodiment, at least the step of reacting a keratin material with a protease and a reducing agent may occur in a closed system (e.g. in a sealed vessel or reactor).

One advantage of the claimed process is that it allows for a single enzymatic step to produce keratin (e.g. feather) hydrolyzate at a lower temperature (e.g. 50 to 80° C.) under reduced conditions compared with traditional processes which employ 120-133° C. (e.g. for 20 to 90 minutes). Suitably, the keratin hydrolyzate produced by the present invention may have a higher nutritional value. This may in part be due to the reduced loss of amino acids which are unstable at high temperatures (e.g. lysine, tryptophan, threonine and tyrosine) and/or the reduced loss of amino acids which can be oxidised (e.g. methionine).

In one aspect, the level of oxygen is controlled such that the oxygen level in the air space of the reactor is less than 50% of the air, preferably less than 30% of the air, preferably less 5% of air in the reactor.KERATIN MATERIAL

Keratin is a fibrous structural protein found in feather, hair, fur, hooves, nails, wool, claws and scales. Keratins can be divided into two separate groupsαkeratins and β-keratins. The β-keratins are generally harder than α-keratins as they contain more cysteine linkages.

The keratin material for use in the methods and uses of the present invention can be any substance which comprises keratin. Suitably, the keratin material may comprise feathers, hair, fur, hooves, nails and wool.

In one embodiment, the keratin material comprises β-keratin. β-keratins may be found in nails and claws of reptile, shells of Testudines and in the feathers beaks and claws of birds and porcupines. Suitably, the keratin material may be feathers, preferably poultry feathers, preferably chicken feathers.

In one embodiment, the keratin material comprises α-keratin. α-keratins may be found in the hair (including wool), horns, nails, claws and hooves of mammals.

In one embodiment, the keratin material comprises α-keratin. α-keratins may be found in the hair (including wool), horns, nails, claws and hooves of mammals. Suitably, the keratin material may be hair, horns or hooves.

In one embodiment, the keratin material comprises feathers, hair (such as pig hair), horns, hooves or wool. Suitably, the keratin material may be feathers, hair, horns, hooves or wool.

In one embodiment, at least 5 g of keratin material is used in the processes and uses of the present invention. Suitably, at least 50 g, preferably at least 100 g, preferably at least 500 g keratin material is used in the processes and uses of the present invention.

In one embodiment, at least 1 kg f keratin material is used, preferably at least 2 kg, or at least 10 kg, or at least 20 kg, or at least 30 kg keratin material is used.

Protese

The term “protease” as used herein is synonymous with peptidase or proteinase.

The protease for use in the present invention may be a subtilisin (E.C. 3.4.21.62) or a bacillolysin (E.C. 3.4.24.28) or an alkaline serine protease (E.C. 3.4.21.x) or a keratinase (E.C. 3.4.x.x). Suitably, a protease for use in the present invention may be an Protease endopeptidase K (EC 3.4.2.1.64), pronase, papain, an endopeptidase Arg-C, an endoprotease Gluc-C (EC 3.4.21.19), an enterokinase (EC 3.4.21.9), a collagenase (EC 3.4.24.3), a thermolysin (EC 3.4.24.27), a trypsin (EC 3.4.21.4), a chymotrypsin (EC 3.4.21.1), a pepsin (EC 3.4.23.1), an aspergillopepsin (EC 3.4.23.18), a sedolisin (EC 3.4.21.100), or a dipeptidyl peptidase (EC 3.4.14.1).

Preferably the protease in accordance with the present invention is a subtilisin, a serine protease, a metalloprotease, an acid protease, a neutral protease or a keratinase.

Suitable proteases include those of animal, vegetable or microbial origin. Chemically modified or protein engineered mutants are also suitable. The protease may be a serine protease or a metalloprotease, e.g., an alkaline microbial protease or a trypsin-like protease. Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115. All of which are incorporated herein by reference.

The terms “wild type protease enzyme” or “wild type” in accordance with the invention describe a protease enzyme with an amino acid sequence found in nature.

The terms “protease enzyme variant”, “protease variant” or “variant” in accordance with the invention describe a protease enzyme with an amino acid sequence derived from the amino acid sequence of a parent protease but differing by one or more amino acid substitutions, insertions, and/or deletions, which together are referred to as “mutations”. It is envisaged that a protease enzyme variant may also be a parent protease enzyme for further rounds of methods of preparing protease variants such as molecular evolution.

The term “homologous polypeptide(s)”, according to the present invention, described also as “homologues” herein, describe polypeptides, preferably protease enzymes (i.e. “homologous protease ” or “homologous enzymes”) with a sequence identity of more than 75% compared to a first polypeptides/proteases/enzymes amino acid sequence, preferably has at least 80%, 85%, 90%, 95%, 96%, 97%, 98% or 99% sequence homology. The term “functional equivalent thereof means that the enzyme has to have about the same functional characteristics as that of the protease detailed herein. The term “modified form” or “variant” means that the enzyme has been modified from its original form but retains the same enzymatic functional characteristics. In particular, the terms “variant” or “modified form” encompass protease enzymes with an amino acid sequence derived from the amino acid sequence of the parent/wild-type protease and having one or more amino acid substitutions, insertions, and/or deletions, which together are referred to as mutations. Modified forms or variants may display altered enzyme characteristics compared to the parent enzyme. Preferably, modified forms or variants have one or more of the following enhanced phenotypes: increased thermostability and/or; an increased proteolytic (for example pepsin) stability and/or; an increased specific activity and/or; broader substrate specificity and/or; an activity over a broader pH range. The term “functional” or “effective” fragment means a fragment or portion of the protease that retains about the same enzymatic function or effect.

As used herein the term “thermostable” relates to the ability of the enzyme to retain activity after exposure to elevated temperatures.

As used herein the term “pH stable” relates to the ability of the enzyme to retain activity over a wide range of pH's.

In one preferred embodiment the protease for use in the present invention may be one or more of the proteases in one or more of the commercial products below:

| Commercial | | | |
|---|---|---|---|
| product ® | Company | Protease type | Protease source |
| Protex 30L ™ | Genencor/DuPont | Serine protease* | B. subtilis |
| Protex 6L ™ | Genencor/DuPont | Serine protease* | B. amyloliquefaciens |
| Purafect 4000L ™ | Genencor/DuPont | Serine protease* | |
| FNA ™ | Genencor/DuPont | Serine protease* | B. amyloliquefaciens |
| Properase 1600L ™ | Genencor/DuPont | Serine protease* | B. alcalophilus |
| Protex P ™ | Genencor/DuPont | subtilisin | |
| Esperase 8.0L | Novozymes | protease | Bacillus sp. |
| Everlase 16.0 ™ | | subtilisin | Bacillus sp. |
| Alcalase 2.4 ™ | Novozymes | subtilisin | Bacillus sp. |
| Neutrase 0.8L ™ | Novozymes | protease | B. amyloliquefaciens |
| Allzyme FD ™ | Alltech | Serine protease* | Aspergillus niger |
| Arazyme One-Q ™ | Insect Biotech Co. | metalloprotease | Serratia proteamaculans |
| | | | HY-3 |
| Savinase ™ | Novozymes | subtilisin | Bacillus sp. |
| Ronozyme ProAct | DSM/Novozymes | Alkaline serine | Nocardiopsis prasina |
| | | protease | gene expressed in |
| | | | Bacillus licheniformis |
| Versazyme/Cibenz | Novus | Keratinase | Bacillus licheniformis |
| a DP100 | | | |

Additionally or in the alternative the protease may be comprised in one or more of the following commercially available products: Kannase™ NovoCarne Tender™ and Novozym 37020, Novo-Pro D™ (all available from Novozymes); BioSorb-ACDP™ (Noor Creations, India); or Angel™ Acid Protease (Angel Yeast Co, Ltd., China).

Suitably, the protease may be a protease from Bacillus (such as Bacillus subtilis, Bacillus amyloliquefaciens, B. alcalophilus and B. licheniformis), Trichoderma, Nocardiopsis, Serratia or Aspergillus.

In one embodiment, the protease is from Bacillus. Suitably, the protease may be from the species Bacillus subtilis, Bacillus amyloliquefaciens, B. Alcalophilus, B. lentus and B. licheniformis. In one embodiment, the protease is from the species Bacillus subtilis.

Amino Acid Sequences

In one embodiment, the protease has the amino acid sequence ID No.1.

In one embodiment, the protease has the amino acid sequence ID No.2.

The scope of the present invention also encompasses amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The protein encompassed in the present invention may be used in conjunction with other proteins, particularly enzymes. Thus the present invention also covers a combination of proteins wherein the combination comprises the protease of the present invention and another enzyme, which may be another protease according to the present invention.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

Sequence Identity or Sequence Gomology

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in percentage homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum percentage homology therefore firstly requires the production of an optimal alignment, taking into consideration gap penalties. A suitable computer program for carrying out such an alignment is the the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate percentage homology, preferably percentage sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 0 |
| GAP EXTENSION | 0 |

| FOR CLUSTAL | DNA | PROTEIN | |
|---|---|---|---|
| WORD SIZE | 2 | 1 | K triple |
| GAP PENALTY | 15 | 10 | |
| GAP EXTENSION | 6.66 | 0.1 | |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence may be determined over the whole sequence.

Variants/Homologues/Derivatives

The present invention also encompasses the use of variants, homologues and derivatives of any amino acid sequence of a protein or of any nucleotide sequence encoding such a protein.

Here, the term “homologue” means an entity having a certain homology with the subject amino acid sequences and the subject nucleotide sequences. Here, the term “homology” can be equated with “identity”.

In the present context, a homologous sequence is taken to include an amino acid sequence which may be at least 75, 80, 85 or 90% identical, preferably at least 95, 96, 97, 98 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 80, 85 or 90% identical, preferably at least 95, 96, 97, 98 or 99% identical to a nucleotide sequence encoding an enzyme of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate percentage homology between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in percentage homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p 387). Examples of other software than can perform sequence comparisons include, but are not limited to, the BLAST package (see Ausubel et aL, 1999 Short Protocols in Molecular Biology, 4^{th} Ed—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244).

Once the software has produced an optimal alignment, it is possible to calculate percentage homology, preferably percentage sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Norwell D C, Trends Biotechnol. (1995) 13(4), 132-134.

In one aspect, preferably any of the protease sequences according to the present invention is in an isolated form. The term “isolated” means that the protease sequence is at least substantially free from at least one other component with which the protease sequence is naturally associated in nature and as found in nature. The protease sequence of the present invention may be provided in a form that is substantially free of one or more contaminants with which the substance might otherwise be associated. Thus, for example it may be substantially free of one or more potentially contaminating polypeptides and/or nucleic acid molecules.

In one aspect, preferably the protease sequence according to the present invention is in a purified form. The term “purified” means that the a given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

In one embodiment, the protease is encoded by the nucleic acid sequence ID No.3.

In one embodiment, the protease is encoded by the nucleic acid sequence ID No.4.

The scope of the present invention encompasses nucleotide sequences encoding proteins having the specific properties as defined herein. The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand. The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

Preparation of the Nucleotide Sequence

A nucleotide sequence encoding either a protein which has the specific properties as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

Molecular Evolution

As a non-limiting example, it is possible to produce numerous site directed or random mutations into a nucleotide sequence, either in vivo or in vitro, and to subsequently screen for improved functionality of the encoded polypeptide by various means.

In addition, mutations or natural variants of a polynucleotide sequence can be recombined with either the wildtype or other mutations or natural variants to produce new variants. Such new variants can also be screened for improved functionality of the encoded polypeptide. The production of new preferred variants can be achieved by various methods well established in the art, for example the Error Threshold Mutagenesis (WO 92/18645), oligonucleotide mediated random mutagenesis (U.S. Pat. No. 5,723,323), DNA shuffling (U.S. Pat. No. 5,605,793), exo-mediated gene assembly WO00/58517. The application of these and similar random directed molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following:

optimised expression and/or activity in a host cell or in vitro,

increased enzymatic activity, altered substrate and/or product specificity,

increased or decreased enzymatic or structural stability, altered enzymatic

activity/specificity in preferred environmental conditions, e.g.

temperature, pH, substrate

Site-Directed Mutagenesis

Once a protein-encoding nucleotide sequence has been isolated, or a putative protein-encoding nucleotide sequence has been identified, it may be desirable to mutate the sequence in order to prepare a protein of the present invention.

Mutations may be introduced using synthetic oligonucleotides. These oligonucleotides contain nucleotide sequences flanking the desired mutation sites.

A suitable method is disclosed in Morinaga et al., (Biotechnology (1984) 2, p 646-649). Another method of introducing mutations into enzyme-encoding nucleotide sequences is described in Nelson and Long (Analytical Biochemistry (1989), 180, p 147-151).

Recombinant

In one aspect the sequence for use in the present invention is a recombinant sequence—i.e. a sequence that has been prepared using recombinant DNA techniques.

These recombinant DNA techniques are within the capabilities of a person of ordinary skill in the art. Such techniques are explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press.

In one aspect the sequence for use in the present invention is a synthetic sequence—i.e. a sequence that has been prepared by in vitro chemical or enzymatic synthesis. It includes, but is not limited to, sequences made with optimal codon usage for host organisms—such as the methylotrophic yeasts Pichia and Hansenula.

Use of Enzyme

Preferably, the protease is used in range of about 1 g/kg of keratin material to about 50 g/kg keratin material.

In one embodiment, the keratin material comprises (or consists of) feathers and the protease is used in range of about 1 g/kg of keratin material to about 10 g/kg keratin material.

In one embodiment, the keratin material comprises (or consists of) wool, horns, hooves or admixtures thereof and the protease is used in range of about 1 g/kg of keratin material to about 50 g/kg keratin material.

It will be understood that one protease unit (PU) is the amount of enzyme that liberates from the substrate (0.6% casein solution) one microgram of phenolic compound (expressed as tyrosine equivalents) in one minute at pH 7.5 (40 mM Na₂PO₄/lactic acid buffer) and 40° C. This may be referred to as the assay for determining 1PU.

In one embodiment suitably the enzyme as a subtilisin (E.C. 3.4.21.62) or a bacillolysin (E.C. 3.4.24.28) or an alkaline serine protease (E.C. 3.4.21.x) or a keratinase (E.C. 3.4.x.x) and the E.C. classification designates an enzyme having that activity when tested in the assay taught herein for determining 1 PU.

In one embodiment, the protease is an alkaliphile and optimally hydrolyses at a pH between about pH 7 to about pH 12. Suitably, the protease may optimally hydrolyse at a pH between about pH 8 to about pH 11. Suitably, the protease may optimally hydrolyse at a pH between about pH 8 to about pH 10. Suitably, the protease may optimally hydrolyse at a pH of about 9.

In one embodiment, the protease is an acidophile and optimally hydrolyses at a pH between about pH 1 to about pH 7. Suitably, the protease may optimally hydrolyse at a pH between about pH 3 to about pH 6. Suitably, the protease may optimally hydrolyse at a pH between about pH 4 to about pH 5.

In one embodiment, the protease is a neutrophile and optimally hydrolyses at a pH between about pH 6 to about pH 8. Suitably, the protease may optimally hydrolyse at a pH of about 7.

In one embodiment, the protease may hydrolyse optimally at a temperature between about 30° C. to about 90° C. Suitably, the protease may hydrolyse optimally at a temperature between about 40° C. to about 80° C. Suitably, the protease may hydrolyse optimally at a temperature between about 50° C. to about 80° C. Preferably, the protease may hydrolyse optimally at a temperature between about 60° C. to about 80° C.

Reducing Agent

The term “reducing agent” as used herein (also referred to as a reductant or reducer) refers to an element or compound in a reduction-oxidation (redox) reaction that donates an electron to another species. Thus a reducing agent will be oxidised in a redox reaction.

The presence of a reducing agent may stimulate keratin degradation by a protease. Without wishing to be bound by theory it is thought that reducing agents may breakdown disulphide bonds present in keratin, opening up the structure to aid hydrolysis by a protease.

A substance “stimulates” keratin degradation if it increases the speed by which a desired level of keratin degradation is reached and/or if it increases the amount of digestible protein and/or amount of amino acids available after a set time (e.g. 8 hours).

Suitably, a reducing agent may be added prior to and/or during the step of reacting keratin material with a protease and a reducing agent.

In one embodiment, only one reducing agent is used in combination with a protease in the methods and uses of the present invention. In another embodiment, any combination of two or more reducing agents are used. For example any combination of 2 or 3 or 4 or 5 or 6 or 7 or 8 or 9 or 10 surfactants may be used.

In one embodiment, one or more reducing agent(s) may be selected from the group consisting of: salts of sulphite (e.g. Na₂SO₃ and NaHSO₃), bisulfite, dithionite metabisulfite sulphur dioxide, DTT, β-mercaptoethanol and sulphide.

Suitably, the reducing agent may be sodium sulphite or sodium bisulphite.

Whilst one or more reducing agents may be added in the methods and uses of the present invention, the present inventors have surprisingly found that by controlling oxygen levels the amount of reducing agent used to achieve a desired degree of keratin degradation may be reduced.

In one embodiment, the reducing agent may be continuously added during the reaction of the keratin material with a protease. Advantageously, continuous addition of a reducing agent during the reaction or a series of additions of the reducing agent (e.g. multiple dosing) during the reaction may result in improved enzymatic hydrolysis. Without wishing to be bound by theory, it is thought that the reducing agent may be oxidised during the reaction leading to a depletion of reducing agent to break down the disulphide bonds in the keratin material. Therefore, multiple dosing or continuous addition of the reducing agent may allow a more linear reaction of keratin hydrolysis during the enzymatic reaction. Furthermore, multiple dosing and/or continuous addition of the reducing agent may allow more control over the levels of the reducing agent present at the end of the reaction. This may provide safety advantages as the process can be controlled to ensure lower amounts of the reducing agent in final product. Thus, suitably the reducing agent added may be continuously added or may be added in multiple doses (such as 2 or more times, or 3 or more times, or 4 or more times, or 5 or more times or 10 or more times).

Further Components

Suitably, the keratin material and protease and/or chemical may optionally be admixed (simultaneously or sequentially) with one or more further components.

Examples of further components include surfactants, additional enzymes, chemicals, antimicrobials, metal ions in the form of a salt, carriers, excipients, diluents, fats, peptides and minerals.

In one embodiment one or more further components may be selected from the group consisting of: surfactants, chemicals, additional enzymes, metal ions in the form of a salt, carriers, excipients, diluents, fats, peptides, minerals and combinations thereof

In one embodiment, one or more additional enzyme(s) are added. Said one or more additional enzymes may be selected from the group consisting of esterases, lipases, cutinases, protein-disulfide reductases (EC 1.8.x.x), metalloproteases, aspartic acid proteases, cysteine proteases, exopeptidases, endoproteases, acyltransferases, perhydrolases, oxidases (e.g. hexose oxidases and maltose oxidoreductases), and proteases.

In one embodiment, one or more one or more metal ions may be used in the form of a salt. Suitably, the metal ion may be one of the group consisting of Cu, Mg, Mn, Co, Zn, Fe and Ca. Suitably, the salt may be a chloride.

In one embodiment, an antimicrobial is added as a further component. Suitably, sulphite or its salts are added as an antimicrobial

Surfactant

The method and uses of the present invention may additionally utilise a surfactant (e.g. a non-sulphur containing surfactant).

In one embodiment, the term “surfactant” as used herein refers to a substance which reduces the surface tension of a liquid in which it is dissolved. For example, the surfactant may reduce the surface tension of water. The surfactant may preferably act as a detergent, wetting agent, emulsifier or dispersant. Suitably, the surfactant may be amphiphilic (i.e. may contain both hydrophobic and hydrophilic groups.

In another embodiment, the “surfactant” may be an “emulsifier”. The term “emulsifier” as used herein refers to substances which stabilise an emulsion by increasing its kinetic stability.

Preferably, the surfactant (e.g. emulsifier) used is not toxic to animals and/or humans.

Preferably the surfactant is selected from the group consisting of: sodium decanoate; Triton X-100; Tween 20; Tween 80; lecithin; polyoxyethylene stearate; polyoxyethylene sorbitan monolaurate; polyoxyethylene sorbitan monooleate; polyoxyethylene sorbitan monopalmitate; polyoxyethylene sorbitan monostearate; polyoxyethylene sorbitan tristearate; ammonium phosphatides; sodium, potassium or calcium salts of fatty acids; magnesium salts of fatty acids; acetic acid esters of mono- and diglycerides of fatty acids; lactic acid esters of mono- and diglycerides of fatty acids; citric acid esters of mono- and diglycerides of fatty acids; mono- and diacetyl tartaric acid esters of mono- and diglycerides of fatty acids; sucrose esters of fatty acids; sucroglycerides; polyglycerol esters of fatty acids; polyglycerol polyricinoleate; propane-1,2-diol esters of fatty acids; thermally oxidised soya bean oil interacted with mono- and diglycerides of fatty acids; sodium stearoyl-2-lactylate; calcium stearoyl-2-lactylate; sorbitan monostearate; sorbitan tristearate; sorbitan monolaurate; sorbitan monooleate and sorbitan monopalmitate.

In one embodiment, the surfactant is selected from the group consisting of: sodium decanoate; Triton X-100; Tween 20 and Tween 80. Preferably, the surfactant is sodium decanoate.

In one embodiment, the surfactant is an emulsifier selected from the group consisting of: sodium decanoate; Triton X-100; Tween 20; Tween 80; lecithin; polyoxyethylene stearate; polyoxyethylene sorbitan monolaurate; polyoxyethylene sorbitan monooleate; polyoxyethylene sorbitan monopalmitate; polyoxyethylene sorbitan monostearate; polyoxyethylene sorbitan tristearate; ammonium phosphatides; sodium, potassium or calcium salts of fatty acids; magnesium salts of fatty acids; acetic acid esters of mono- and diglycerides of fatty acids; lactic acid esters of mono- and diglycerides of fatty acids; citric acid esters of mono- and diglycerides of fatty acids; mono- and diacetyl tartaric acid esters of mono- and diglycerides of fatty acids; sucrose esters of fatty acids; sucroglycerides; polyglycerol esters of fatty acids; polyglycerol polyricinoleate; propane-1,2-diol esters of fatty acids; thermally oxidised soya bean oil interacted with mono- and diglycerides of fatty acids; sodium stearoyl-2-lactylate; calcium stearoyl-2-lactylate; sorbitan monostearate; sorbitan tristearate; sorbitan monolaurate; sorbitan monooleate and sorbitan monopalmitate. Advantageously, such surfactants are emulsifiers that are currently used in the food industry and are generally recognised as safe for use in food.

In one embodiment, only one surfactant is used in combination with a protease in the methods and uses of the present invention. In another embodiment, any combination of two or more surfactants are used. For example any combination of 2 or 3 or 4 or 5 or 6 or 7 or 8 or 9 or 10 surfactants may be used.

The optimal amount of surfactant to be used can be readily determined by a person of ordinary skill in the art.

In one embodiment, the amount of surfactant used may be in the range of about 0.01% w/v to about 1% w/v. Preferably, the amount of surfactant may be in the range of about 0.05-0.9% w/v.

Suitably, the amount of surfactant used may be greater than or equal to 0.01% w/v keratin material. Preferably, the amount of surfactant used may be greater than or equal to 0.02% w/v; or 0.05% w/v or 0.1% w/v keratin material.

Suitably, the amount of surfactant used may be less than or equal to 1% w/v keratin material. Preferably, the amount of surfactant used may be less than or equal to 0.9% w/v; or 0.8% w/v or 0.5% w/v keratin material.

In one embodiment, the amount of surfactant used may be in the range of about 0.1 g/Kg to about keratin material 10 g/Kg keratin material (e.g. feathers). Suitably, the amount of surfactant used may be in the range of about 0.5 g/Kg to about keratin material 5 g/Kg keratin material (e.g. feathers).

Suitably, the amount of surfactant used may be greater than or equal to 0.1 g/Kg keratin material. Preferably, the amount of surfactant used may be greater than or equal to 0.5 g/Kg; or 1 g/Kg; or 2 g/Kg keratin material.

Suitably, the amount of surfactant used may be less than or equal to 10 g/Kg keratin material. Preferably, the amount of surfactant used may be less than or equal to 8 g/Kg; or 5 g/Kg or 3 g/Kg keratin material.

In one embodiment, the ratio of surfactant to keratin material may be in the range of about 1:100 to about 1:10,000. Preferably the ratio of surfactant to keratin material may be in the range of about 1:500 to about 1:5,000.

Suitably, the ratio of surfactant to keratin material may be greater than 1:10,000.

Suitably, the ratio of surfactant to keratin material may be less than 1:100.

In one embodiment, the surfactant is a non-sulphur containing surfactant. Without wishing to be bound by theory, it is believed that the non-sulphur containing surfactant may work by opening up the hydrophobic keratin material in the solution thereby allowing the enzyme better access to the structure to carry out the hydrolysis. Alternatively the surfactant may help to remove hydrolyzed protein from the surface of the feather to solution so that the underlying surface of the feather is exposed to proteolysis.

In one embodiment, a non-sulphur containing surfactant may be a surfactant for use in the methods and uses of the present invention if in a process for degrading a keratin material (e.g. feathers) there is an increase in the amount of soluble protein present in solution at the end of the process compared to the amount of soluble protein present in solution for a control method in which the non-sulphur containing surfactant was not used. Suitably, the increase in soluble protein may be measured by an increase in absorbance at 215-280 nm.

Chemical Hydrolysis

In one embodiment of the present invention, the enzymatic hydrolysis of keratin is combined with chemical hydrolysis.

Methods for the chemical hydrolysis of keratin are known in the art.

Advantageously, the method of hydrolysing or degrading keratin using a protease and a non-sulphur containing surfactant can result in a reduction of the time and/or amount of chemicals needed using convention chemical hydrolysis methods.

The present inventors have surprisingly found that the combination of enzymatic and chemical hydrolysis of keratin can result in a quick and efficient process for keratin degradation.

In one aspect the chemical hydrolysis step occurs prior to, during and/or after the reaction step with the protease and non-sulphur containing surfactant.

Suitably, the chemical reaction may occur prior to and/or during step the enzymatic reaction and the chemical adjusts the pH to a desirable working pH of the protease.

In one embodiment, the pH used for the chemical reaction is a pH under which the protease works.

In one embodiment, the protease used is an alkaliphile and the chemical oxidant is an alkali. In another embodiment, the protease used is an acidophile and the chemical oxidant is an acid.

In another embodiment, the chemical softens the keratin material by disrupting the three dimensional structure of keratin (e.g. by disrupting the hydrogen bonding, salt bridges, and/or hydrophilic and hydrophobic interactions of the keratin structure).

Suitably step ii) may occur after step i).

Suitably, a chemical (such as an acid, alkali or oxidant) may be used to chemically hydrolyse keratin. Without wishing to be bound by theory, it is believed that chemical oxidants work by oxidising the disulphide bridges present in keratin which may result in the solubilisation of the keratin. Advantageously, the use of a chemical oxidant may also result in the keratin hydrolyzate been soluble in water which may provide easy of application to e.g. feed.

The term “solubility” as used herein refers to the ability of hydrolysed keratin to dissolve in a solvent (e.g. in water).

The solubility of the keratin hydrolyzate can be measured by determining the amount of nitrogen in the supernatant following centrifugation of the reaction mixture.

The term “chemical oxidant” as used herein refers to a substance that removes electrons from another reactant in a redox chemical reaction. Chemical oxidants are also referred to as oxidising agents or oxidants.

Examples of chemical oxidants which can hydrolyse keratin include sodium chlorite, HCl, acetic acid, hydroxyacetic acid, NaOH, peracids, HOCl, HOBr, NaClO₂, ClO₂, H₂O₂, ammonium hydroxide, sodium hydroxide, and calcium hydroxide.

As used herein the term “a peracid” (also known as a peroxy acid or peroxyacid) is an acid which contains an acidic —OOH group. The two main classes of peracids are those derived from conventional mineral acids, especially sulfuric acid, and the organic derivatives of carboxylic acids. Generally peracids are known to be strong oxidisers.

The general formula of a peracid is shown below:

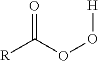

Suitably the peracid may be peracetic acid or performic acid.

In one embodiment, peracetic acid may be generated in reaction medium. This can be achieved by the treatment of acetic acid with hydrogen peroxide using sulfuric acid as the catalyst: H₂O₂+CH₃CO₂H [symbol] CH₃CO₃H+H₂O. Hydrogen peroxide can be generated enzymatically by for example glucose oxidase and hexose oxidase. Peracetic acid can also be generated by the reaction of triacetin with H₂O₂ catalyzed by perhydrolase or aryl esterase.

In one embodiment, the chemical may be generated in the reaction medium.

The keratin material and chemical oxidant are admixed until a desired degree of degradation of the keratin material has occurred (e.g. until the digestibility of keratin material is increased, such as by enriching the concentration of digestible proteins and peptides therein). A person of ordinary skill in the art will readily understand that the optimal time period used will depend on a number of factors such as the temperature and pH used; the degree of cross-linking present in the keratin material to be degraded; and whether other components are used in the process.

When the enzymatic hydrolysis occurs prior to the chemical hydrolysis, reference to admixing the chemical with “keratin material” refers to admixing the chemical with the resultant material following treatment of keratin material with a protease. Thus, the keratin material which is admixed with the chemical may be partially hydrolysed or degraded. Suitably, the keratin material may be in the form of a precipitate.

In one embodiment the chemical is used at a concentration of less than 50 mM, Suitably, the chemical is used at a concentration of less than 40 mM, preferably less than 30 mM, preferably less than 20 mM. Suitably, the chemical may be used at a concentration of less than 10 mM.

In one embodiment, the chemical is used at a concentration of between about 0.1 mM to about 49 mM. Suitably, the chemical may be used at a concentration of between about 0.2 mM and about 40 mM, preferably at concentration between about 0.5 and about 10 mM.

In one embodiment, the keratin material and chemical oxidant may be admixed for about 5 minutes to about 24 hours or from about 10 minutes to about 20 hours or from about 15 minutes to about 16 hours or from about 30 minutes to about 10 hours or from about 1 hour to about 8 hours or from about 3 to about 7 hours.

In another embodiment, the keratin material and chemical may be admixed for about 30 min to 48 hours.

In one embodiment, the keratin material and chemical may be admixed for about 1 hour to about 8 hours.

Suitably the keratin material and the chemical oxidant may be admixed for at least 5 minutes or at least 10 minutes or at least 15 minutes or at least 20 minutes or at least 25 minutes or at least 30 minutes or at least 45 minutes or at least 1 hour or at least 1.5 hours or at least 2 hours or at least 2.5 hours or at least 3 hours or at least 3.5 hours or at least 4 hours or at least 4.5 hours or at least 5 hours or at least 5.5 hours or at least 6 hours or at least 7 hours or at least 8 hours or at least 9 hours or at least 10 hours.

In one embodiment, the keratin material and the chemical may be admixed for at least 1 hour.

Suitably, the keratin material and the chemical oxidant may be admixed for less than 24 hours, or less than 20 hours, or less than 16 hours, or less than 12 hours or less than 10 hours, or less than 8 hours, or less than 6 hours or less than 4 hours, or less than 2 hours, or less than 1 hour.

In one embodiment, the keratin material and the chemical may be admixed for less than 10 hours.

In one embodiment, the keratin material and the chemical oxidant may be admixed until at least 50% by weight of the keratin material is degraded. Suitably, at least 60%, or at least 70%, or at least 80%, or at least 90% or at least 95% or 100% by weight of the keratin material is degraded.

A combination of chemical and enzymatic hydrolysis is not generally used in the commercial production of keratin hydrolyzate or the commercial degradation of keratin. This is primarily due to the increased costs associated with this combination. For example, Kim et al. (Poultry Science, 2002, 81:95-98) disclose that whilst that a combination of 24 h enzyme treatment and 2-hour chemical treatment were significantly more than double the costs of enzyme treatment or chemical treatment alone.

Advantageously, the present inventors have surprisingly found that enzymatic and chemical hydrolysis may be combined in a way which significantly reduces the costs typically associated with the combined use of enzymatic and chemical hydrolysis.

Suitably the pH during the reaction will depend on the chemical oxidant used. For example, NaOH works at alkaline pH and HCl as acid pH.

Suitably the temperature during the reaction may be adjusted to optimise chemical degradation of the keratin material.

In one embodiment, when chemical hydrolysis occurs, preferably the chemical hydrolysis step with a chemical occurs at a time point in the process which avoids additional costs associated with the adjustment of the pH for the enzymatic reaction.

If the protease is an alkaliphile, preferably the chemical is an alkali and the chemical reaction occurs prior to or simultaneously with the enzymatic reaction. If the protease is an acid, preferably the chemical is an acid and the chemical reaction occurs prior to or simultaneously with the enzymatic reaction. If the protease is an alkaliphile and the chemical is an acid, preferably, the enzymatic reaction occurs prior to the chemical reaction so that the chemical reaction brings down the pH. If the protease is an acioiphile and the chemical is an alkali, preferably, the enzymatic reaction occurs prior to the chemical reaction so that the chemical reaction brings up the pH. In this way, additional costs associated with raising or lowering the pH to optimal conditions for the protease are avoided.

Suitably, where the chemical hydrolysis step occurs prior to and/or during (e.g. simultaneously with) the reaction with protease, the reaction conditions used are adapted to the working ranges of the protease used.

In one aspect of the present invention, the chemical used for the chemical hydrolysis also adjusts the pH to the optimal working conditions of the protease, thereby advantageously providing the benefit of combined chemical and enzymatic reactions whilst minimising or avoiding additional costs associated with combining a chemical hydrolysis step with an enzymatic hydrolysis. For example, the protease Protex 30L works under high alkaline conditions. Thus, this protease may be combined with an alkali known to chemically hydrolyse keratin such as NaOH. Advantageously, the alkali then works to adjust the pH to the working range of the protease whilst also degrading keratin itself. In this way chemical and enzymatic hydrolysis can be combined in a cost-effective way. Clearly, a protease which works under acidic conditions can be combined with an acid known to degrade keratin such as HCl in a similar way.

In one embodiment, the methods of the present invention may use an acidophilic protease. In this embodiment, the methods of the present invention may comprise a chemical hydrolysis step prior to and/or during the reaction with protease, wherein the chemical hydrolysis step uses an acid.

In one embodiment, the methods of the present invention may use an alkaliphilic protease. In this embodiment, the methods of the present invention may comprise a chemical hydrolysis step prior to and/or during the reaction with protease, wherein the chemical hydrolysis step uses an alkali.

Combining chemical hydrolysis with enzymatic hydrolysis may allow for a reduction of the concentration of the chemical used and/or duration which the keratin material is exposed to the chemical may be reduced to achieve a desired level of keratin degradation. Thus, keratin hydrolyzate may be produced which advantageously has an enhanced protein digestibility and/or increased source of amino acids compared to the use of either enzymatic or chemical hydrolysis alone.

In one aspect, protein digestibility may be measured by measuring Kjeldahl N content (e.g. in a N autoanalyzer) of the supernatant, following centrifugation of the reaction mixture.

In one aspect, “increased source of amino acids” refers to an increase in in vitro amino acid digestibility which may be measured in accordance with Kim et al. (Poultry Science, 2002, 85:95-98) using the following equation:

$$100 - \frac{\begin{matrix}amino acid content ( g \text{/} 100 g reaction precipitate ) \\ of treatment \times ( 100 ) - N solubility of treatment )\end{matrix}}{\begin{matrix}amino acid content ( g \text{/} 100 g reaction precipitate ) \\ of control \times ( 100 ) - N solubility of control )\end{matrix}} \times 100$$

In one aspect, the chemical hydrolysis step may occur after the reaction step with a protease. This may be advantageous in situations where the protease selected and the chemical oxidant selected does not work at overlapping pH.

Process for Preparing a Feedstuff

In another aspect there is provided a method for producing animal feed comprising admixing keratin hydrolyzate produced by a method of the present invention with one or more animal feed constituents.

Advantageously, keratin hydrolyzate produced in accordance with a method of the present invention may provide a valuable source of protein and/or source of amino acids in animal feed. For example, keratin hydrolyzate can provide a source of one or more of the following amino acids: methionine, cysteine, lysine, threonine, arginine, isoleucine, leucine, valine, histidine, phenylalanine, glycine, serine, proline, alanine, aspartic acid and glutamic acid.

The terms “animal feed” and “feedstuff” are used interchangeably herein. The terms “animal feed constituents” and “feed ingredients” are also used interchangeably.

Animal feed is typically produced in feed mills in which raw materials are first ground to a suitable particle size and then mixed with appropriate additives. The animal feed may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of the animal feed may also involve an additional step that includes extrusion or expansion prior to pelleting—in particular by suitable techniques that may include at least the use of steam.

By way of example only animal feed for chickens, e.g. broiler chickens, may be comprised of one or more of the ingredients listed in the table below, for example in the percentages given in the table below:

| Ingredients | Starter (%) | Finisher (%) |
|---|---|---|
| Maize | 46.2 | 46.7 |
| Wheat Middlings | 6.7 | 10.0 |
| Maize DDGS | 7.0 | 7.0 |
| Soyabean Meal 48% CP | 32.8 | 26.2 |
| An/Veg Fat blend | 3.0 | 5.8 |
| L-Lysine HCl | 0.3 | 0.3 |
| DL-methionine | 0.3 | 0.3 |
| L-threonine | 0.1 | 0.1 |
| Salt | 0.3 | 0.4 |
| Limestone | 1.1 | 1.1 |
| Dicalcium Phosphate | 1.2 | 1.2 |
| Poultry Vitamins and Micro- | 0.3 | 0.3 |
| minerals | | |

By way of example only the diet specification for chickens, such as broiler chickens, may be as set out in the table below:

| Diet specification | | |
|---|---|---|
| Crude Protein (%) | 23.00 | 20.40 |
| Metabolizable Energy Poultry (kcal/kg) | 2950 | 3100 |
| Calcium (%) | 0.85 | 0.85 |
| Available Phosphorus (%) | 0.38 | 0.38 |
| Sodium (%) | 0.18 | 0.19 |
| Dig. Lysine (%) | 1.21 | 1.07 |
| Dig. Methionine (%) | 0.62 | 0.57 |
| Dig. Methionine + Cysteine (%) | 0.86 | 0.78 |
| Dig. Threonine (%) | 0.76 | 0.68 |

By way of example only a feedstuff suitable for consumption by laying hens may comprise of one or more of the ingredients listed in the table below, for example in the percentages given in the table below:

| Ingredient | Laying phase (%) |
|---|---|
| Maize | 10.0 |
| Wheat | 53.6 |
| Maize DDGS | 5.0 |
| Soybean Meal 48% CP | 14.9 |
| Wheat Middlings | 3.0 |
| Soybean Oil | 1.8 |
| L-Lysine HCl | 0.2 |
| DL-methionine | 0.2 |
| L-threonine | 0.1 |
| Salt | 0.3 |
| Dicalcium Phosphate | 1.6 |
| Limestone | 8.9 |
| Poultry Vitamins and Micro-minerals | 0.6 |

By way of example only the diet specification for laying hens may be as set out in the table below:

| Diet specification | |
|---|---|
| Crude Protein (%) | 16.10 |
| Metabolizable Energy Poultry (kcal/kg) | 2700 |
| Lysine (%) | 0.85 |
| Methionine (%) | 0.42 |
| Methionine + Cysteine (%) | 0.71 |
| Threonine (%) | 0.60 |
| Calcium (%) | 3.85 |
| Available Phosphorus (%) | 0.42 |
| Sodium (%) | 0.16 |

By way of example only a feedstuff for turkeys may comprise one or more of the ingredients listed in the table below, for example in the percentages given in the table below:

| | Phase 1 | Phase 2 | | Phase 4 |
|---|---|---|---|---|
| Ingredient | (%) | (%) | Phase 3 (%) | (%) |
| Wheat | 33.6 | 42.3 | 52.4 | 61.6 |
| Maize DDGS | 7.0 | 7.0 | 7.0 | 7.0 |
| Soyabean Meal 48% CP | 44.6 | 36.6 | 27.2 | 19.2 |
| Rapeseed Meal | 4.0 | 4.0 | 4.0 | 4.0 |
| Soyabean Oil | 4.4 | 4.2 | 3.9 | 3.6 |
| L-Lysine HCl | 0.5 | 0.5 | 0.4 | 0.4 |
| DL-methionine | 0.4 | 0.4 | 0.3 | 0.2 |
| L-threonine | 0.2 | 0.2 | 0.1 | 0.1 |
| Salt | 0.3 | 0.3 | 0.3 | 0.3 |
| Limestone | 1.0 | 1.1 | 1.1 | 1.0 |
| Dicalcium Phosphate | 3.5 | 3.0 | 2.7 | 2.0 |
| Poultry Vitamins and | 0.4 | 0.4 | 0.4 | 0.4 |
| Micro-minerals | | | | |

By way of example only the diet specification for turkeys may be as set out in the table below:

Diet specification

| | | | | |
|---|---|---|---|---|
| Crude Protein (%) | 29.35 | 26.37 | 22.93 | 20.00 |
| Metabolizable Energy Poultry | 2.850 | 2.900 | 2.950 | 3.001 |
| (kcal/kg) | | | | |
| Calcium (%) | 1.43 | 1.33 | 1.22 | 1.02 |
| Available Phosphorus (%) | 0.80 | 0.71 | 0.65 | 0.53 |
| Sodium (%) | 0.16 | 0.17 | 0.17 | 0.17 |
| Dig. Lysine (%) | 1.77 | 1.53 | 1.27 | 1.04 |
| Dig. Methionine (%) | 0.79 | 0.71 | 0.62 | 0.48 |
| Dig. Methionine + Cysteine (%) | 1.12 | 1.02 | 0.90 | 0.74 |
| Dig. Threonine (%) | 1.03 | 0.89 | 0.73 | 0.59 |

By way of example only a feedstuff for piglets may comprise one or more of the ingredients listed in the table below, for example in the percentages given in the table below:

| Ingredient | Phase 1 (%) | Phase 2 (%) |
|---|---|---|
| Maize | 20.0 | 7.0 |
| Wheat | 25.9 | 46.6 |
| Rye | 4.0 | 10.0 |
| Wheat middlings | 4.0 | 4.0 |
| Maize DDGS | 6.0 | 8.0 |
| Soyabean Meal 48% CP | 25.7 | 19.9 |
| Dried Whey | 10.0 | 0.0 |
| Soyabean Oil | 1.0 | 0.7 |
| L-Lysine HCl | 0.4 | 0.5 |
| DL-methionine | 0.2 | 0.2 |
| L-threonine | 0.1 | 0.2 |
| L-tryptophan | 0.03 | 0.04 |
| Limestone | 0.6 | 0.7 |
| Dicalcium Phosphate | 1.6 | 1.6 |
| Swine Vitamins and Micro-minerals | 0.2 | 0.2 |
| Salt | 0.2 | 0.4 |

By way of example only the diet specification for piglets may be as set out in the table below:

| Diet specification | | |
|---|---|---|
| Crude Protein (%) | 21.50 | 20.00 |
| Swine Digestible Energy (kcal/kg) | 3380 | 3320 |
| Swine Net Energy (kcal/kg) | 2270 | 2230 |
| Calcium (%) | 0.80 | 0.75 |
| Digestible Phosphorus (%) | 0.40 | 0.35 |
| Sodium (%) | 0.20 | 0.20 |
| Dig. Lysine (%) | 1.23 | 1.14 |
| Dig. Methionine (%) | 0.49 | 0.44 |
| Dig. Methionine + Cysteine (%) | 0.74 | 0.68 |
| Dig. Threonine (%) | 0.80 | 0.74 |

By way of example only a feedstuff for grower/finisher pigs may be comprises of one or more of the ingredients listed in the table below, for example in the percentages given in the table below:

| Ingredient | Grower/Finisher (%) |
|---|---|
| Maize | 27.5 |
| Soyabean Meal 48% CP | 15.4 |
| Maize DDGS | 20.0 |
| Wheat bran | 11.1 |
| Rice bran | 12.0 |
| Canola seed meal | 10.0 |
| Limestone | 1.6 |
| Dicalcium phosphate | 0.01 |
| Salt | 0.4 |
| Swine Vitamins and Micro-minerals | 0.3 |
| Lysine-HCl | 0.2 |
| Vegetable oil | 0.5 |

By way of example only the diet specification for grower/finisher pigs may be as set out in the table below:

| Diet specification | |
|---|---|
| Crude Protein (%) | 22.60 |
| Swine Metabolizable Energy (kcal/kg) | 3030 |
| Calcium (%) | 0.75 |
| Available Phosphorus (%) | 0.29 |
| Digestible Lysine (%) | 1.01 |
| Dig. Methionine + Cysteine (%) | 0.73 |
| Digestible Threonine (%) | 0.66 |

Thus, it can be seen that keratin hydrolyzate can be used as a good and potentially cheap source of protein and/or amino acids required in these diets.

Feed

When used in the preparation of a feedstuff, the keratin hydrolyzate produced in accordance with the present invention may be used in conjunction with one or more of: a nutritionally acceptable carrier, a nutritionally acceptable diluent, a nutritionally acceptable excipient, a nutritionally acceptable adjuvant or a nutritionally active ingredient.

The term “animal feed” as used herein means food suitable for animal consumption, such as for cows, pigs, lamb, sheep, goats, chickens, turkeys, ostriches, pheasants, deer, elk, reindeer, buffalo, bison, antelope, camels, kangaroos; horses, fish; cats, dogs, guinea pigs, rodents e.g. rats, mice, gerbils and chinchillas.

The keratin hydrolyzate produced in accordance with the present invention may be added to the animal feed or a component in a manner known per se.

Preferably the feed may be a fodder, or a premix thereof, a compound feed, or a premix thereof. In one embodiment keratin hydrolyzate produced in accordance with the present invention may be admixed with, and/or applied onto, a compound feed, a compound feed component or to a premix of a compound feed or to a fodder, a fodder component, or a premix of a fodder.

The term fodder as used herein means any food which is provided to an animal (rather than the animal having to forage for it themselves). Fodder encompasses plants that have been cut.

The term fodder includes hay, straw, silage, compressed and pelleted feeds, oils and mixed rations, and also sprouted grains and legumes.

Fodder may be obtained from one or more of the plants selected from: alfalfa (lucerne), barley, birdsfoot trefoil, brassicas, Chau moellier, kale, rapeseed (canola), rutabaga (swede), turnip, clover, alsike clover, red clover, subterranean clover, white clover, grass, false oat grass, fescue, Bermuda grass, brome, heath grass, meadow grasses (from naturally mixed grassland swards, orchard grass, rye grass, Timothy-grass, corn (maize), millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include corn, soybeans, sorghum, oats, and barley.

Suitably a premix as referred to herein may be a composition composed of microingredients such as vitamins, minerals, chemical preservatives, inhibitory substances, fermentation products, and other essential ingredients. Premixes are usually compositions suitable for blending into commercial rations.

In the method of preparing an animal feed in accordance with the present invention one or more animal feed constituents may be added selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

Animal feed produced by the method of the present invention may contain at least 30%, at least 40%, at least 50% or at least 60% by weight corn and soybean meal or corn and full fat soy, or wheat meal or sunflower meal.

In addition or in the alternative, animal feed produced by a method of the present invention may comprise at least one high fibre feed material and/or at least one by-product of the at least one high fibre feed material to provide a high fibre feedstuff. Examples of high fibre feed materials include: wheat, barley, rye, oats, by products from cereals, such as corn gluten meal, Distillers Dried Grain Solubles (DDGS), wheat bran, wheat middlings, wheat shorts, rice bran, rice hulls, oat hulls, palm kernel, and citrus pulp. Some protein sources may also be regarded as high fibre: protein obtained from sources such as sunflower, lupin, fava beans and cotton.

In the present invention the feed may be one or more of the following: a compound feed and premix, including pellets, nuts or (cattle) cake; a crop or crop residue: corn, soybeans, sorghum, oats, barley, corn stover, copra, straw, chaff, sugar beet waste; fish meal; freshly cut grass and other forage plants; meat and bone meal; molasses; oil cake and press cake; oligosaccharides; conserved forage plants: hay and silage; seaweed; seeds and grains, either whole or prepared by crushing, milling etc.; sprouted grains and legumes; yeast extract.

As used herein the term “applied” refers to the indirect or direct application of the keratin hydrolyzate produced in accordance with the present invention to the product (e.g. the feed). Examples of the application methods which may be used, include, but are not limited to, treating the product in a material comprising the keratin hydrolyzate, direct application by mixing the keratin hydrolyzate with the product, spraying the keratin hydrolyzate onto the product surface or dipping the product into a preparation of the keratin hydrolyzate.

In one embodiment the keratin hydrolyzate produced by a method of the present invention is preferably admixed with, or applied onto, the product (e.g. feedstuff). Alternatively, the keratin hydrolyzate may be included in the emulsion or raw ingredients of a feedstuff.

As used herein the term “swine” relates to non-ruminant omnivores such as pigs, hogs or boars. Typically, swine feed includes about 50 percent carbohydrate, about 20 percent protein and about 5% fat. An example of a high energy swine feed is based on corn which is often combined with feed supplements for example, protein, minerals, vitamins and amino acids such as lysine and tryptophan. Examples of swine feeds include animal protein products, marine products, milk products, grain products and plant protein products, all of which may further comprise natural flavourings, artificial flavourings, micro and macro minerals, animal fats, vegetable fats, vitamins, preservatives or medications such as antibiotics. It is to be understood that where reference is made in the present specification, including the accompanying claims, to ‘swine feed’ such reference is meant to include “transition” or “starter” feeds (used to wean young swine)and “finishing” or “grower” feeds (used following the transition stage for growth of swine to an age and/or size suitable for market).

As used herein the term “poultry” relates to fowl such as chickens, broilers, hens, roosters, capons, turkeys, ducks, game fowl, pullets or chicks. Poultry feeds may be referred to as “complete” feeds because they contain all the protein, energy, vitamins, minerals, and other nutrients necessary for proper growth, egg production, and health of the birds. However, poultry feeds may further comprise vitamins, minerals or medications such as coccidiostats (for example Monensin sodium, Lasalocid, Amprolium, Salinomycin, and Sulfaquinoxaline) and/or antibiotics (for example Penicillin, Bacitracin, Chlortetracycline, and Oxytetracycline).

Young chickens or broilers, turkeys and ducks kept for meat production are fed differently from pullets saved for egg production. Broilers, ducks and turkeys have larger bodies and gain weight more rapidly than do the egg-producing types of chickens. Therefore, these birds are fed diets with higher protein and energy levels.

It is to be understood that where reference is made in the present specification, including the accompanying claims, to “poultry feed” such reference is meant to include “starter” feeds (post-hatching),“finisher”,“grower” or “developer” feeds (from 6-8 weeks of age until slaughter size reached) and “layer” feeds (fed during egg production).

Pet Food

In one aspect, the “animal feed” may be a pet food. The term “pet food” as used herein means a food suitable for consumption by a domesticated animal such as a dog, cat, horse, pig, fish, bird, hamster, gerbil, guinea pig, rodent e.g. rat, mouse, rabbit and chinchilla

The keratin hydrolyzate may be applied on, or in, the pet food itself and/or constituent(s) (e.g. ingredients) of the pet food. For example, the keratin hydrolyzate may be applied on, or in, a palatant.

Examples of typical constituents found in dog and cat food include palatants, Whole Grain Corn, Soybean Mill Run, Chicken By-Product Meal, Powdered Cellulose, Corn Gluten Meal, Soybean Meal, Chicken Liver Flavor, Soybean Oil, Flaxseed, Caramel Color, Iodized Salt, L-Lysine, Choline Chloride, Potassium Chloride, vitamins (L-Ascorbyl-2-Polyphosphate (source of vitamin C), Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Folic Acid, Vitamin D3 Supplement), Vitamin E Supplement, minerals (e.g., Ferrous Sulfate, Zinc Oxide, Copper Sulfate, Manganous Oxide, Calcium Iodate, Sodium Selenite), Taurine, L-Carnitine, Glucosamine, Mixed Tocopherols, Beta-Carotene, Rosemary Extract.

A pet food recipe suitable for addition of the keratin hydrolysate of the current invention may be based on the following main ingredients: maize meals (whole, meal, or ground), poultry edible offal meal, wheat bran, alfalfa meal, maize gluten, rice, linseed meal, rapeseed meal, soybean meal or bean meal. Preferably this recipe also contains stabilizers, and is supplemented with various vitamins and antioxidants such as vitamin a, cholecalciferol, vitamin E, menadione, citric acid, pantothenic acid, folic acid, vitamin B12, vitamin B6, riboflavin (vitamin B2), vitamin B1, niacin (vitamin B3), and preferably flavour enhancers such as glutamine, taurine, yeast extract, and salt. Alternatively, or additionally, said recipe may be supplemented with a reconstituted food ingredient such as beef meal, powdered beef bone, chicken fat, chicken liver (hydrolyzed), fish meal or fish powder (such as tuna powder).

In one aspect, the pet food may be a wet or dry pet food, which may be in the form of a moist pet food (e.g. comprising 18-35% moisture or event 18-70% moisture), semi-moist pet food (e.g. 14 to 18% moisture), dry pet food, pet food supplement or a pet treat. Some pet food forms (e.g. moist and semi-moist pet food) are particularly susceptible to contamination due to the fact that the processing conditions for preparing the pet food are not sufficient to kill all microorganisms on, or in, the pet food.

Suitably, the pet food may be in kibble form.

In one aspect, the pet food may be suitable for a dog or a cat.

In one aspect the pet food prepared with said keratin hydrolysate of the current invention may be described as anallergeinc if it assists pets that usually experience adverse food reactions to the extent that the animals can be described as having food allergies or intolerances, and in the worst cases inflammatory bowel disease. A typical anallergeinc pet food composition usually maintains the standard 20% protein composition, which can be in the form of keratin hydrolysate if the pepsin digestability is sufficiently high (ideally greater than 75%), and also contains such innocuous ingredients as corn starch, coconut oil, soybean oil or hydrolysed soy, natural flavours, potassium phosphate, powdered cellulose, calcium carbonate, sodium silico aluminate, chicory, L-tyrosine, fructooligosaccharides, fish oil, L-lysine, choline chloride, taurine, L-tryptophan, vitamins [DL-alpha tocopherol (source of vitamin E), inositol, niacin, L-ascorbyl-2-polyphosphate (source of vitamin C), D-calcium panthotenate, biotin, pyridoxine hydrochloride (vitamin B6), riboflavine (vitamin B2), thiamine mononitrate (vitamin B1), vitamin A acetate, folic acid, vitamin B12 supplement, vitamin D3 supplement], DL-methionine, marigold extract (Tagetes erecta L.), histidine, trace minerals [zinc proteinate, zinc oxide, ferrous sulfate, manganese proteinate, copper proteinate, copper sulfate, manganous oxide, calcium iodate, sodium selenite], rosemary extract, preserved with natural mixed tocopherols and citric acid.

In one aspect, the pet food may be fish food. A fish food normally contains macro nutrients, trace elements and vitamins necessary to keep captive fish in good health. Fish food may be in the form of a flake, pellet or tablet. Pelleted forms, some of which sink rapidly, are often used for larger fish or bottom feeding species. Some fish foods also contain additives, such as beta carotene or sex hormones, to artificially enhance the color of ornamental fish.

In one aspect, the pet food may be a bird food. Bird food includes food that is used both in birdfeeders and to feed pet birds. Typically bird food is comprised of a variety of seeds, but may also encompass suet (beef or mutton fat).

In one aspect, the keratin hydrolyzate may be incorporated within the pet food or on the surface of the pet food, such as, by spraying or precipitation thereon.

In one aspect, the keratin hydrolyzate is formulated for use in pet food. In this aspect, the keratin hydrolyzate may comprise additional anti-contaminant agents such as phosphoric acid, propionic acid and propionates, sulfites, benzoic acid and benzoates, nitrites, nitrates and parabens.

Suitably, the keratin hydrolyzate may be added to a pet food or constituent thereof such that the keratin hydrolyzate is present at about 0.1% to about 10%, about 0.1 to about 5%, or about 0.1 to about 3% by weight of the pet food. In one aspect the anti-contaminant composition is present at about 0.1, 0.2, 0.3, 0.4, 0.5, 0.6, 0.7, 0.8, 0.9, 1.0, 1.1, 12., 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, or 5.0% by weight of the pet food where any of the stated values can form an upper or lower endpoint when appropriate.

In one aspect, the pet food may be a kibble. An illustrative method of preparing a kibble comprises the following steps:

a. preconditioning by mixing wet and dry ingredients at elevated temperature to form a kibble dough;

b. extruding the kibble dough at a high temperature and pressure;

c. drying the extruded kibble; and

d. enrobing or coating the dried kibble with topical liquid and/or dry ingredients.

Suitably, the keratin hydrolyzate can be applied to the kibble at any stage in the process, such as at step a and/or d.

Forms

The keratin hydrolyzate produced by the method of the present invention may be used in any suitable form—whether when alone or when present in a composition. Said composition may include other nutrition-rich waste streams from slaughter houses such as blood or meat and bone meal, or it may be prepared in a composition with other animal feeds including but not limited to soybean meal, fish meal, fish oil, whey powder, whey filtrate, distillers grains, cottonseed meal, corn gluten meal, canola meal, and the like.

The dry powder or granules may be prepared by means known to those skilled in the art, such as, in top-spray fluid bed coater, in a buttom spray Wurster or by drum granulation (e.g. High sheer granulation), extrusion, pan coating or in a microingredients mixer.

Suitably, the keratin hydrolyzate may be provided as a spray-dried or freeze-dried powder. An example of such a spray dried keratin hydrolysate is shown in example 4. Such spray dried powders have the advantages of increased stability and handling compared to forms with a higher water content, but said powders are still able to be provided in solution or suspended form to young animals most in need of nutritional supplement. In one embodiment of the current invention the spray dried keratin hydrolysate is in a powder form especially suitable for feeding young pets and commercial livestock, including but not limited to cows, pigs, mink, dogs, cats, broiler chickens and turkeys. The small particle size of the spray dried keratin hydrolysate also makes it particularly suitable for smaller creatures such as young fish (fry), and crustaceans such as shrimp and crab or crab larvae.

In one aspect, the keratin hydrolyzate is in a liquid formulation. Such liquid consumption may contain one or more of the following: a buffer, salt, sorbitol and/or glycerol.

In one embodiment the keratin hydrolyzate of the present invention may formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

Isolated

In one aspect, suitably the enzyme(s) used in the present invention may be in an isolated form. The term “isolated” means that the enzyme is at least substantially free from at least one other component with which the enzyme is naturally associated in nature and as found in nature. The enzyme of the present invention may be provided in a form that is substantially free of one or more contaminants with which the substance might otherwise be associated. Thus, for example it may be substantially free of one or more potentially contaminating polypeptides and/or nucleic acid molecules.

Purified

In one aspect, preferably the enzyme according to the present invention is in a purified form. The term “purified” means that the enzyme is present at a high level. The enzyme is desirably the predominant component present in a composition. Preferably, it is present at a level of at least about 90%, or at least about 95% or at least about 98%, said level being determined on a dry weight/dry weight basis with respect to the total composition under consideration.

The invention will now be described, by way of example only, with reference to the following Figures and Examples.

### EXAMPLES

### Example 1

Experimental: To all 6 flasks with a total volume of 300 ml, 3 g (1% w/v) feathers, sodium sulfite 0.3 g (0.1% w/v), and Protex P, 3 ml (final 1%) were added, followed by the addition of one magnetic bar to each flask. Flasks 1-3 were each filled with 300 ml tricine-NaOH buffer (pH8.5) warmed up to 50° C., and closed loosely with lock, while flasks 4-6 were each filled with 300 ml deaerated tricine-NaOH buffer (pH8.5) and closed tightly with the lock. Deaeration was achieved by heating the solution to 100° C. for around 5 min to expel dissolved air. All flasks were incubated at 50° C. in a water bath with a magnetic stirring at 360 rpm. After 22 hr the reaction mixture were stored at 5° C. for analysis.

Analysis: 100 microliter each of the supernatant from each flask were taken and centrifuged at 15000×g for 15 min. The supernatant following centrifugation was transferred to a microplate with UV transparent bottom for the measurement of optical density at 280 nm as an indication of aromatic amino acids and peptides released in the soluble fraction.

The average absorbance for flasks 1 to 3 was 1.424 whilst the average absorbance for flasks 4 to 6 was 1.518.

In a repeat experiment using the same hydrolysis conditions but a centrifugation step of 10000 rpm for 5 minutes, the OD280 nm values for Flask 1-3 measured was 1.274 (standard deviation, 0.029), whilst the values for Flasks 4-6 was 1.378 (standard deviation, 0.054).

Therefore, it can be seen that in a deaerated stirred reactor, the UV absorbance at 280 nm was higher than that of the reactor that were not deaerated. Results with other proteases confirm this observation, This indicates that the keratin hydrolysis reactions performed by the proteases under lower oxygen levels achieved by heating the buffer used in the reaction and by having the reactors airtight closed (Flasks 4-6) in the presence of sodium sulfite as the reducing agent, gave better hydrolysis than the same proteases performing hydrolysis reactions in aerated conditions.

### Example 2

To each of 6 of 13 ml plastic tubes 100 mg ground chicken feather (the rachis and hollow shafts are shorter than 0.5 cm by the grinding), 5 mg sodium bisulfite, and 65 microliter Protex P was added. To tubes 1-3 12 ml tap water pH adjusted to pH 8.64 were added whereas to tubes 4-6 were 12 ml tap water pH adjusted to pH 8.64 were added and deaerated (by bubbling with nitrogen gas). The tubes were closed tightly and incubated at 50° C. with shaking at 130 rpm for 16 h. The 6 tubes were then centrifuged at 4000 rpm and 0.2 ml of the supernatant obtained from each samples was filtered through a 0.45 micron filter and 50 microliter of the filtrate were measured at 280 nm as an indication for released peptides (see table 1 below). From the table 1 below, one can see that deaeration by nitrogen bubbling of the reaction medium improved the peptide release by 6.5%.

TABLE 1

| | | | | | | | | Standard |
|---|---|---|---|---|---|---|---|---|
| | Absorbance (280 nm) | | | | | | average | deviation |
| Tube | 0.343 | 0.336 | 0.335 | 0.34 | 0.338 | 0.325 | 0.336 | 0.006 |
| 1-3 | | | | | | | | |
| Tube | 0.379 | 0.371 | 0.342 | 0.345 | 0.361 | 0.347 | 0.358 | 0.015 |
| 4-6 | | | | | | | | |

### Example 3

Protex 6L (also called FoodPro alkaline protease) and Protex P are two subtilisin type proteases from DuPont. Protex 6L has a pH range of 7-10, optimum pH is pH 9.5, temperature range 26-70° C., optimal temperature is 60° C.; Protex P has optimal pH range of 7.5-11, optimum is 8.5, optimal temperature is 70° C. In this example a comparison of the two alkaline subtilisin type proteases (EC 3.4.21.62) Protex 6L and Protex P for their hydrolysis of 4 substrates are given. From Table 2 below it can be seen that Protex 6L had high activity on the soluble tetrapeptide substrate AAPF (N-succinyl-ala-ala-pro-phe-p-nitroanalide) which is a standard highly sensitive assay substrate for subtilisin proteases. Protex P on the other hand had higher activity on the insoluble substrates including cross-linked casein (Azurine-casein is a commercial endo-protease substrate), chicken feather and wool keratin.

TABLE 2
A comparison of Protex 6L and Protex P on
different on the hydrolysis of different substrates

| | | | Standard |
|---|---|---|---|
| Proteases | Substrates | Activities for different substrates | deviation |
| Protex 6L | AAPF | 45 (mOD at 410 nm/min)¹⁾ | 2.4 |
| Protex P | AAPF | 10 (mOD at 410 nm/min)¹⁾ | 0.9 |
| Protex 6L | Azurine-casein | 1.032 (OD590 nm)²⁾ | 0.049 |
| Protex P | Azurine-casein | 1.624 (OD590 nm)²⁾ | 0.018 |
| Protex 6L | Chicken | 21.25 (Protein concentration, | 0.46 |
| | feather | mg/10 ml)³⁾ | |
| Protex P | Chicken | 26.75 (Protein concentration, | 3.33 |
| | feather | mg/10 ml)³⁾ | |
| Protex 6L | Wool keratin | 26.8 (Residual keratin left in | 0.2 |
| | | mg)⁴⁾ | |
| Protex P | Wool keratin | 26.8 (Residual keratin left in | 0.6 |
| | | mg)⁴⁾ | |

¹⁾Portions of 100 μl of each of the proteases were diluted to 10 ml (20 mM Mops, pH 7.5), mixed and diluted further with the Mops buffer. The total dilution was 197452 times. The reaction mixture contained 165 μl 0.2M Mops, pH 7.5, 5 μl substrate AAPF (AAPF 1 mg/ml in DMSO from −20° C. was diluted 10 times in water before use) and 20 μl of the diluted protease. OD410 nm was followed spectrophotometrically at 30° C. Activity is given as slope (mOD/min).
²⁾The reaction mixture contained 1 Protazyme Ak tablet from Megazyme (www.megazyme.com), 1.75 ml water, 0.2 ml Tricine-HCl (0.5M, pH 8.5). 50 μl 4450 times diluted protease was added to start the reaction after a pre-incubation at 40° C. for 5 min. The reaction was stopped at 30 min, centrifuged at 4000 rpm for 10 min, the supernatant obtained was measured at 590 nm. In control 50 μl 0.2M Mops buffer was added instead of the protease. Activity was given as the increase in absorbance at 590 nm.
³⁾To 13 ml yellow capped tubes with known weight were added 100 mg chicken feather ground to pieces shorter than 0.5 cm in length (final 1% (w/w), 9.8 ml milli-Q water with pH adjusted to 9.44 using NaOH, and 170 μl 5.88% sodium sulfite so that its final concentration was 0.1%. The reaction was started by the addition of 30 μl each of the protease products (undiluted). The reaction was performed in the closed airtight tube at 60o C. for 8 hours 40 min. The supernatant obtained containing solubilized peptides was assayed for by micro-Kjeldahl nitrogen determination which was converted to protein by a factor of 6.25.
⁴⁾The reaction mixture contained in a 2 ml tube 57 mg wool keratin powder (http://www.tcieurope.eu) in water adjusted to pH 10 with NaOH and 2.5 μl undiluted protease. The reaction was performed at 60° C. for 16 hours with shaking. At the end of the reaction, the unhydrolyzed keratin was collected by centrifugation, dried and weighed as residual keratin.

This example demonstrates the ability of Protex P and Protex 6L to hydrolyse the keratin types found in both wool and feather, which are alpha-keratin and beta-keratin respectively.

### Example 4

40 g chicken feathers (0.33% dry matter) was mixed with 8.4 g 0.5 M Acetic acid/Bis-Tris/CHES/HEPES pH 9 buffer, 1.2 g sodium sulfite with and without 0.6 g Protex P. The two samples were incubated at 65° C. in a rotary mixer for 4 hours. Samples were heated 10 min 100° C. to inactivate the enzyme. The hydrolysate samples were centrifuged (10 min, 10.000×g) and two fractions separated. The soluble fraction (the supernatant) contained soluble peptides, and the pellet fraction (the precipitate) contained insoluble peptides. The pellet fraction was dried in a freeze drier until constant weight was acheived, and weighed. The percent of hydrolyzed feather was 58.8% (standard deviation, 0.98) for the sample including Protex P and 3.8% (standard deviation, 1.6) for the sample without Protex P, calculated as per equation 1.

Hydrolyzed feathers (%)=100%−(X1−X2)/X1*100% Equation 1.

X1=Dry matter mass (g) of chicken feather before hydrolysis

X2=Dry matter mass (g) of pellet fraction

The supernatant from the Protex P hydrolyzed feathers was filtered through a 355 μm screen. A Büchi B-191 mini-spray dryer was first started up and run at stable conditions with ion exchanged water with parameters as Table 3. Once parameters were stable chicken feather hydrolysate was loaded at a feed rate of 30%, and the resulting white powder product collected. The powder had a very light white appearance. The moisture content of the powder was 3.2% (standard deviation, 0.3) measured on an A&D ML-50 moisture analyzer (105° C. drying temperature).

TABLE 3

| | |
|---|---|
| Liquid before spray (° C.) | 20 |
| Inlet Preset (° C.) | 150 |
| Inlet Actual (° C.) | 150 |
| Outlet (° C.) | 98 |
| Aspirator (%) | 100 |
| Pump (%) | 30 |
| Atomizing air flow l/h | 600 |
| Atomizing air inlet, bar | 5 |
| Vacuum (Mbar) | −42 |

### Example 5

Protex P in a formulation with formulation containing glycerol 55% (w/w) and sodium acetate 10% (w/w) with a pH of 5.0 was evaluated for activity toward four different substrates: the standard soluble tetrapeptide substrate AAPF, cross-linked casein, wool keratin, and chicken feather keratin together. Six other commercial alkaline proteases within their standard formulations were similarly tested. Protex P, Protex 6L and 30L were from Du Pont. The alkaline proteases Alcalase 2.4L, Esperase 8L, and Savinase 16L from Novozymes were available commercially. The peptide substrate AAPF was from Bachem AG, wool keratin was from TCI Fine Chemicals, cross-linked casein (Protazyme AK) was from Megazyme. Sodium sulfite was from Sigma-Aldrich (S0505). Chicken feather (Ross 308) was obtained locally in Jutland, Denmark. The feather was cleaned from blood and skins, dried and stored at room temperature (22° C.) before use.

Protease activity on AAPF and cross-linked casein were assayed as follows. Portions of 100 μl of each of the eight products were diluted to 10 ml 20 mM Mops, pH 7.5, mixed and diluted further with 0.2M Mops, pH7.5. The total dilution was 19500 times. The reaction contained 165 μl 0.2M Mops, pH7.5, 5 μl AAPF and 30 μl diluted protease. OD410 nm was followed spectrophotometrically at 30° C. AAPF 1 mg/ml in DMSO from −20° C. was diluted 10× in water before use.

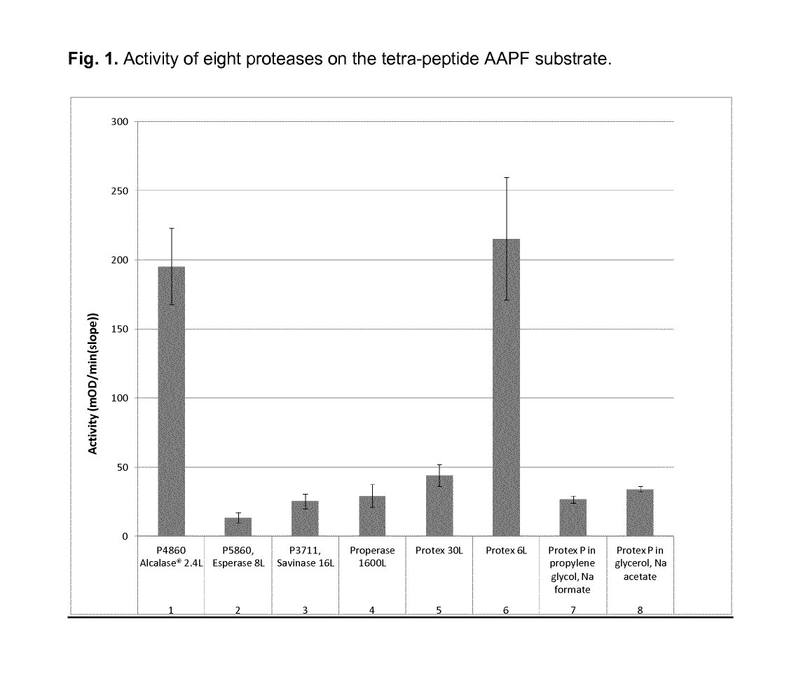

From FIG. 1 it was seen that the Protex P composition formulated with 55% w/w glycerol and 10% w/w sodium acetate had a surprising 26% higher activity based on dosing volume than Protex P in a formulation with 46% w/w propylene glycol and 9.2% w/w sodium formate. FIG. 1 also shows that the activity of Protex P on AAPF is in line with Protex 30L, Properase 1600L, and Savinase 16, where as Protex 6L and Alcalase 2.4L showed up to 10 times higher activity, and Esperase 8L had the lowest activity. This different activity toward AAPF can be due to their substrate specificity and the different concentrations of active proteases, but all of them showed very similar activity towards wool keratin

### Example 6

Eight proteases were tested for performance on cross-linked casein as follows. The reactions contained 1 Protazyme Ak tablet, 1.75 ml water, and 0.2 ml tricine-HCl (0.5M, pH8.5). 50 μl of 4450 times diluted liquid protease was added to start the reaction after a pre-incubation at 40° C. for 5 min. This level of dilution was required because of the very high sensitivity of the assay. The reaction was stopped at 30 min (Test1, n=3) and 40 min (Test2, n=3), centrifuged at 4000 rpm for 10 min before measuring at 590 nm. In the negative control, 50 μl 0.2M Mops buffer was added instead of the protease.

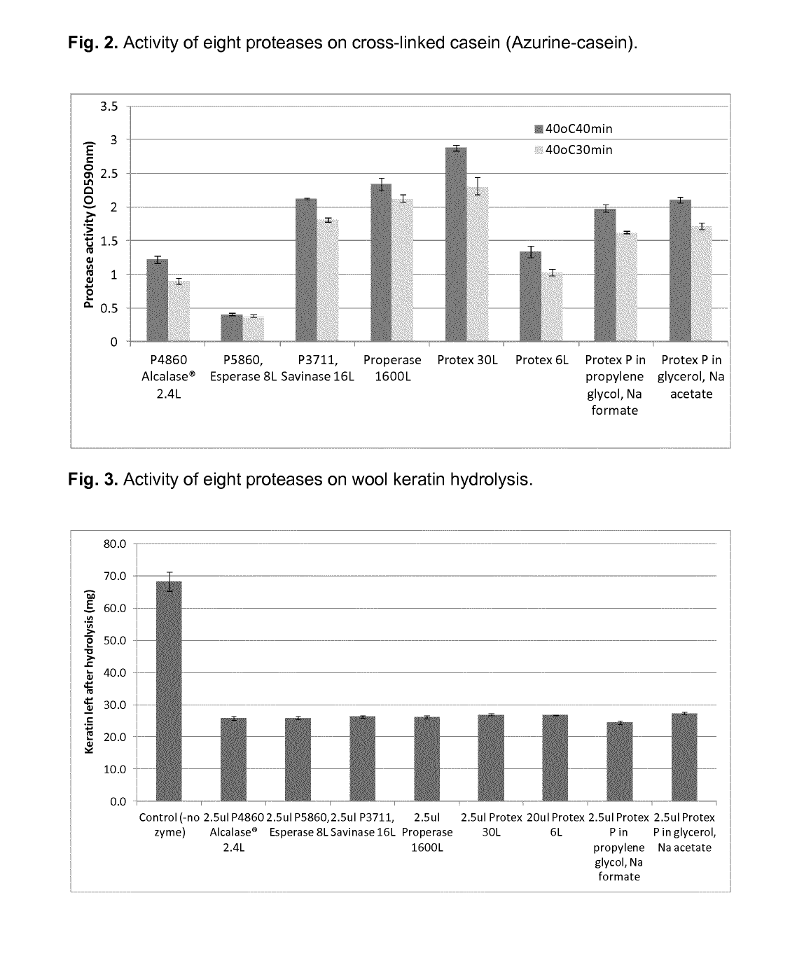

FIG. 2 shows that Protex P in the two different formulations after a dilution of 4450 times in 0.2M Mops-NaOH (pH 7.5) showed essentially the same activity towards cross-linked casein when assayed at two reaction times of 30 and 40 min. Although the assay appears to be outside the linear range from 30 to 40 min, this does not affect the conclusions made herein. Protex 6L and Alcalase 2.4L had the highest activity on AAPF in example 5, but here on cross-linked casein they showed only around half of the activity of Protex P. Protex 30L and Properase 1600L showed the highest activity on cross-linked casein. From FIGS. 1 and 2 it is concluded that these proteases have different activities on the two substrates.

### Example 7

Eight proteases were tested for performance on insoluble wool keratin as follows. The reaction mixture contained in a 2 ml tube 57 mg wool keratin powder (http://www.tcieurope.eu) in water adjusted to pH10 with NaOH and 2.5 μl undiluted protease. The reaction was performed at 60° C. for 16 hours with shaking. At the end of the hydrolysis reaction, the degree of wool keratin hydrolysis was determined by centrifugation to collect the insoluble residual keratin, then dried and weighed.

From FIG. 3 it was seen that all eight alkaline proteases could hydrolyze wool keratin. Based on the residual amount of keratin left after 16 hrs hydrolysis at 60° C., the hydrolysis degree was around 60%, that is, still 40% of the keratin had not been solubilised or hydrolyzed. Adding sulfite was able to promote the wool keratin hydrolysis by an additional 9% (data not shown).

### Example 8

The degree of chicken feather hydrolysis by eight proteases was based on the soluble protein released as determined using the Kjeldahl total nitrogen content method. The reaction volume was 10 ml in a 13 ml airtight tube containing 1% feathers, sulfite 0.1%, tap water, pH9.44. The reaction was started by adding 30 μl of the undiluted protease. No pH adjustment was made during the 9 hr reaction at 60° C. The degree of chicken feather hydrolysis by eight proteases was based on solubilised protein in the supernatant after centrifugation as determined by Kjeldahl method in terms of total nitrogen and converted to protein by a factor of 6.25. The reaction volume was 10 ml in a 13 ml airtight tube containing 1% feathers, sulfite 0.1%, tapp water, pH9.44. The reaction was started by adding 30 μl of the undiluted protease. No pH adjustment was made during the 9 hr reaction at 60° C.

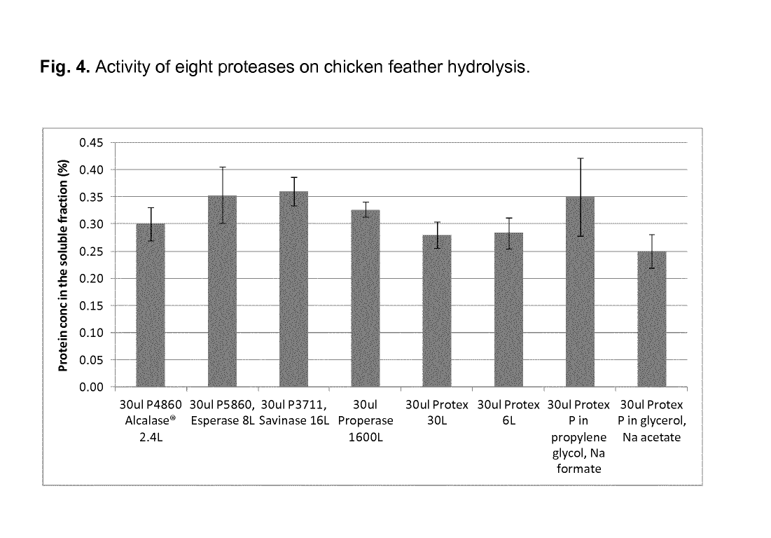

FIG. 4 showed that all eight proteases were able to degrade feather. The protein concentration in the soluble fraction after centrifugation was measured by the Kjeldahl total nitrogen determination.

### Example 9

To 13 ml yellow capped tubes with known weight were added 100 mg ground feather (final 1% (w/w), 9.8 ml milli-Q water with pH adjusted to 9.44 using NaOH, and 170 ul 5.88% sodium sulfite so that final 0.1%. The reaction was started by the addition of 30 ul each of the 3 protease products (undiluted). The feather was either pre-heated at 100° C. for 10 min (H) or not (NH) before being used for the reaction.

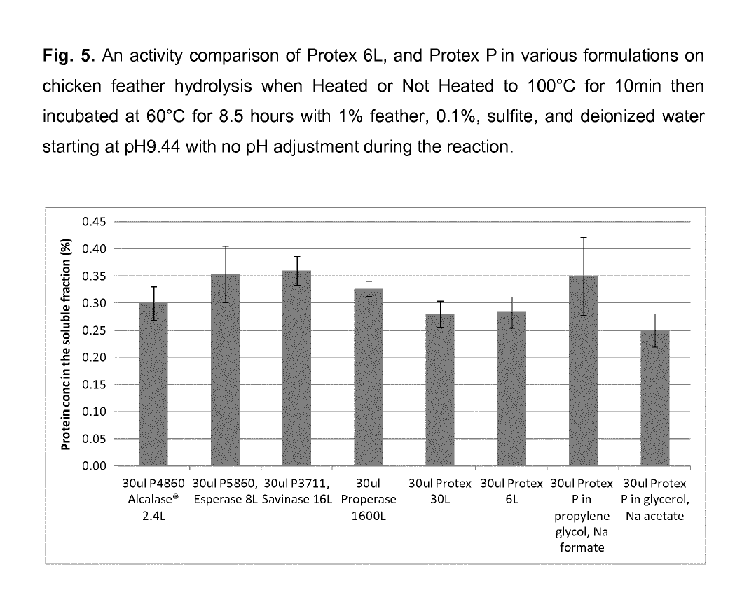

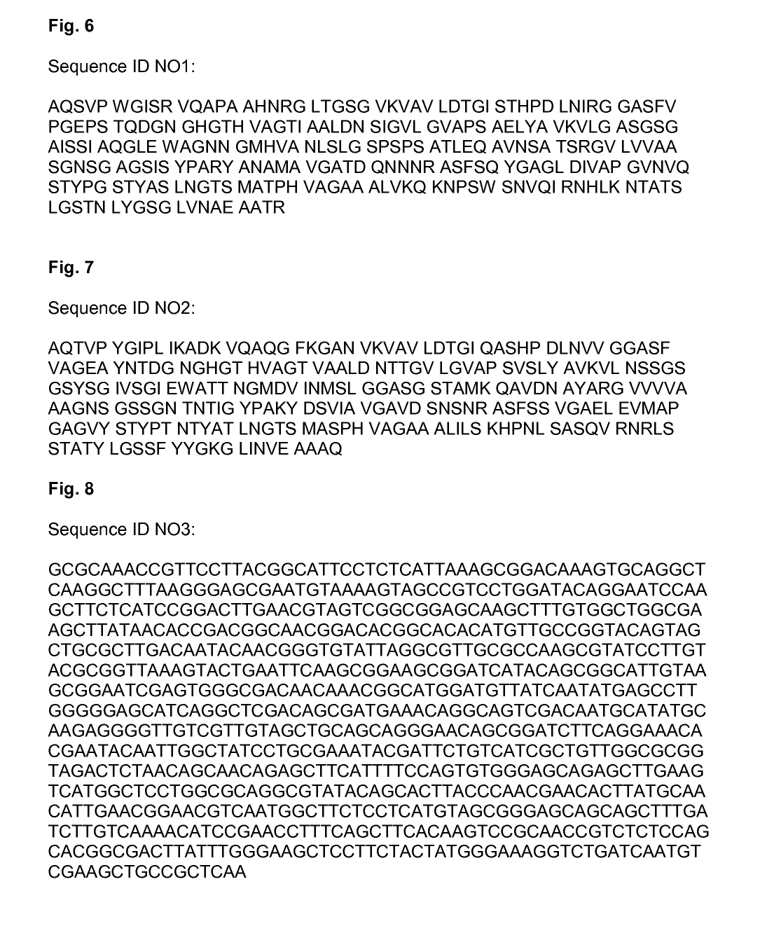

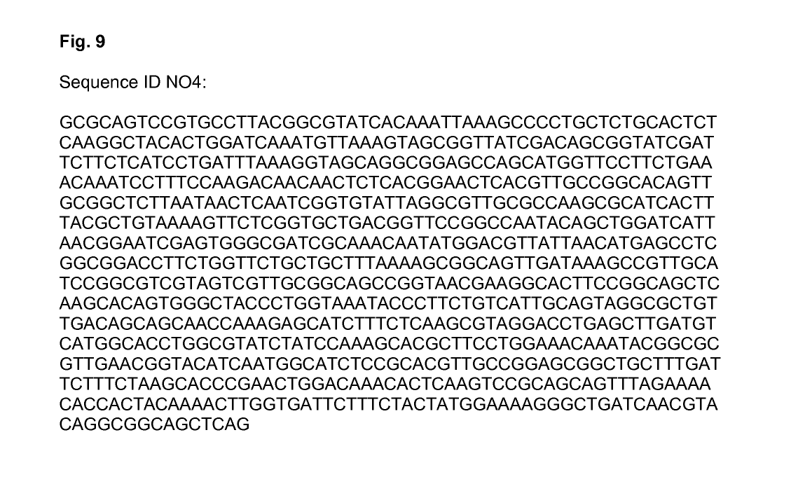

From FIG. 5 it was seen that Protex P in a formulation with propylene glycol and sodium formate performed perhaps slightly better than Protex P in a formulation with glycerol and sodium acetate on chicken feather hydrolysis, either pre-treated at 100° C. for 10 min (H) or untreated (NH). It is also seen that 10 min at 100° C. pre-treatment gave no significant effect with all the three proteases. The reason could be that 10 min pre-treatment compared to a reaction time of 8.5 hours was too short or that 10 min pre-treatment at 100° C. was simply not enough for dry feather.

In general, all eight commercial alkaline proteases showed quite similar hydrolysis activity towards wool keratin and feather as the difference among them was less than 20%.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method of commercially producing keratin hydrolyzate comprising the step of:
i) admixing at least 5 g of keratin material with a protease and a reducing agent under controlled oxygen levels.

2. A method of degrading keratin comprising admixing at least 1 kg keratin material with a protease and a reducing agent under controlled oxygen levels.

3. A method according to claim 1 or claim 2, wherein the reaction occurs in a closed system.

4. A method according to any one of claims 1 to 3, wherein the oxygen levels are controlled by one or more of the followings ways: heating, steam flushing, addition of nitrogen and application of vacuum.

5. A method according to any one of the preceding claims wherein the reducing agent is salts of sulphite (e.g. Na₂SO₃ and NaHSO₃), bisulfite, dithionite metabisulfite, sulphide, sulphur dioxide, DTT and β-mercaptoethanol.

6. A method according to any one of the preceding claims wherein a surfactant is also used in process step i).

7. A method according to claim 6 wherein the surfactant is selected from one or more of the group consisting of: sodium decanoate; Triton-X-100; Tween 20; Tween 80; lecithin; polyoxyethylene stearate; polyoxyethylene sorbitan monolaurate; polyoxyethylene sorbitan monooleate; polyoxyethylene sorbitan monopalmitate; polyoxyethylene sorbitan monostearate; polyoxyethylene sorbitan tristearate; ammonium phosphatides; sodium, potassium or calcium salts of fatty acids; magnesium salts of fatty acids; acetic acid esters of mono- and diglycerides of fatty acids; lactic acid esters of mono- and diglycerides of fatty acids; citric acid esters of mono- and diglycerides of fatty acids; mono- and diacetyl tartaric acid esters of mono- and diglycerides of fatty acids; sucrose esters of fatty acids; sucroglycerides; polyglycerol esters of fatty acids; polyglycerol polyricinoleate; propane-1,2-diol esters of fatty acids; thermally oxidised soya bean oil interacted with mono- and diglycerides of fatty acids; sodium stearoyl-2-lactylate; calcium stearoyl-2-lactylate; sorbitan monostearate; sorbitan tristearate; sorbitan monolaurate; sorbitan monooleate and sorbitan monopalmitate.

8. A method according to any one of the preceding claims wherein the keratin material is comprises one or more of the group consisting of: feathers, hair, fur, hooves, nails and wool.

9. A method according to claim 8 wherein the keratin material comprises feathers.

10. A method according to any one of the preceding claims wherein the protease is from one or more of the following genera selected from the group consisting of: Bacillus, Aspergillus, Streptomyces, Trichoderma, Serratia and Nocardiopsis.

11. A method according to any one of the preceding claims wherein the protease is from the genus Bacillus.

12. A method according to any one of the preceding claims wherein the protease comprises a polypeptide sequence as defined in any one of SEQ ID NOs: 1 or 2, or a functional fragment or variant thereof, having at least 75% sequence identity to any one of SEQ ID NOs: 1 or 2 over at least 50 amino acid residues.

13. A method according to any one of the preceding claims wherein the protease may be transcribed from a nucleic acid sequence encoding a protease with at least 75% identity to either SEQ ID NO3 or SEQ ID NO4 or a nucleic acid sequence capable of hybridizing to the nucleic acid sequence of SEQ ID NO3 or SEQ ID NO4 or the complement thereof under stringent conditions.

14. A method according to any one of the preceding claims wherein the method comprises a chemical hydrolysis step, wherein the chemical hydrolysis occurs prior to, during or after reaction step i).

15. A method according to any one of the preceding claims wherein the keratin material is chopped into smaller pieces prior to step 1).

16. A method of producing animal feed comprising admixing keratin hydrolyzate produced by the method of any one of claims 1 to 13 with one or more animal feed constituents.

17. Use of a protease in combination with a reducing agent in the production of keratin hydrolysate, wherein the protease comprises a polypeptide sequence as defined in any one of SEQ ID NOs: 1 or 2, or a functional fragment or variant thereof having at least 75% sequence identity to any one of SEQ ID NOs: 1 or 2 over at least 50 amino acid residues.

18. Use of a protease in combination with a reducing agent in the production of keratin hydrolysate according to claim 17, wherein the reducing agent is salts of sulphite (e.g. Na₂SO₃ and NaHSO₃), bisulfite, dithionite metabisulfite, sulphide, sulphur dioxide, DTT and β-mercaptoethanol.

19. Use of a protease in combination with a reducing agent in the production of keratin hydrolysate according to claim 17, wherein the protease and reducing agent are admixed with the keratin and the hydrolysis reaction occurs under controlled oxygen conditions.

20. Use of a protease in combination with a reducing agent in the production of keratin hydrolysate according to any one of claims 17 to 19 wherein a surfactant is also admixed with the keratin and the surfactant is selected from one or more of the group consisting of: sodium decanoate; Triton-X-100; Tween 20; Tween 80; lecithin; polyoxyethylene stearate; polyoxyethylene sorbitan monolaurate; polyoxyethylene sorbitan monooleate; polyoxyethylene sorbitan monopalmitate; polyoxyethylene sorbitan monostearate; polyoxyethylene sorbitan tristearate; ammonium phosphatides; sodium, potassium or calcium salts of fatty acids; magnesium salts of fatty acids; acetic acid esters of mono- and and diglycerides of fatty acids; lactic acid esters of mono- and diglycerides of fatty acids; citric acid esters of mono- and diglycerides of fatty acids; mono- and diacetyl tartaric acid esters of mono- and diglycerides of fatty acids; sucrose esters of fatty acids; sucroglycerides; polyglycerol esters of fatty acids; polyglycerol polyricinoleate; propane-1,2-diol esters of fatty acids; thermally oxidised soya bean oil interacted with mono- and diglycerides of fatty acids; sodium stearoyl-2-lactylate; calcium stearoyl-2-lactylate; sorbitan monostearate; sorbitan tristearate; sorbitan monolaurate; sorbitan monooleate and sorbitan monopalmitate.

21. Use of keratin hydrolyzate produced by the method of any one of claims 1 to 13 in feed.

22. Keratin hydrolyzate produced by the method of any one of claims 1 to 13.

23. A feed additive composition comprising the keratin hydrolyzate of claim 16.

24. A feed comprising the keratin hydrolyzate of claim 16.


SEQUENCE LISTING (4 sequences)

SEQ ID NO: 1 - protein, 269 aa, Bacillus licheniformis
AQSVPWGISRVQAPAAHNRGLTGSGVKVAVLDTGISTHPDLNIRGGASFVPGEPSTQDGNGHGTHVAGTIAALDNSIGVLGVAPSAELYAVKVLGASGSGAISSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 274 aa, Bacillus amyloliquefaciens
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 3 - DNA, 822 bp, Bacillus licheniformis
gcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaattggctatcctgcgaaatacgattctgtcatcgctgttggcgcggtagactctaacagcaacagagcttcattttccagtgtgggagcagagcttgaagtcatggctcctggcgcaggcgtatacagcacttacccaacgaacacttatgcaacattgaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaa

SEQ ID NO: 4 - DNA, 825 bp, Bacillus amyloliquefaciens
gcgcagtccgtgccttacggcgtatcacaaattaaagcccctgctctgcactctcaaggctacactggatcaaatgttaaagtagcggttatcgacagcggtatcgattcttctcatcctgatttaaaggtagcaggcggagccagcatggttccttctgaaacaaatcctttccaagacaacaactctcacggaactcacgttgccggcacagttgcggctcttaataactcaatcggtgtattaggcgttgcgccaagcgcatcactttacgctgtaaaagttctcggtgctgacggttccggccaatacagctggatcattaacggaatcgagtgggcgatcgcaaacaatatggacgttattaacatgagcctcggcggaccttctggttctgctgctttaaaagcggcagttgataaagccgttgcatccggcgtcgtagtcgttgcggcagccggtaacgaaggcacttccggcagctcaagcacagtgggctaccctggtaaatacccttctgtcattgcagtaggcgctgttgacagcagcaaccaaagagcatctttctcaagcgtaggacctgagcttgatgtcatggcacctggcgtatctatccaaagcacgcttcctggaaacaaatacggcgcgttgaacggtacatcaatggcatctccgcacgttgccggagcggctgctttgattctttctaagcacccgaactggacaaacactcaagtccgcagcagtttagaaaacaccactacaaaacttggtgattctttctactatggaaaagggctgatcaacgtacaggcggcagctcag